SEQUENCE LISTING (25 sequences)

SEQ ID NO: 1 - protein, 6 aa, Rattus norvegicus
PSRPNR

SEQ ID NO: 2 - protein, 11 aa, Rattus norvegicus
SPTPQRRAPAV

SEQ ID NO: 3 - protein, 17 aa, Rattus norvegicus
RRAPAVPPARPGSRGPA

SEQ ID NO: 4 - protein, 13 aa, Rattus norvegicus
RRSPTSSPTPQRR

SEQ ID NO: 5 - protein, 4 aa, Rattus norvegicus
PAVP

SEQ ID NO: 6 - protein, 30 aa, Rattus norvegicus
PAGRRSPTSSPTPQRRAPAVPPARPGSRGP

SEQ ID NO: 7 - protein, 441 aa, Rattus norvegicus
MSGPYDEASEEITDSFWEVGNYKRTVKRIDDGHRLCNDLMSCVQERAKIEKAYAQQLTDWAKRWRQLIEKGPQYGSLERAWGAMMTEADKVSELHQEVKNSLLNEDLEKVKNWQKDAYHKQIMGGFKETKEAEDGFRKAQKPWAKKMKELEAAKKAYHLACKEEKLAMTREMNSKTEQSVTPEQQKKLVDKVDKCRQDVQKTQEKYEKVLEDVGKTTPQYMEGMEQVFEQCQQFEEKRLVFLKEVLLDIKRHLNLAENSSYIHVYRELEQAIRGADAQEDLRWFRSTSGPGMPMNWPQFEEWNPDLPHTAAKKEKQPKKAEGAALSNATGAVESTSQAGDRGSVSSYDRGQAYATEWSDDESGNPFGGNEANGGANPFEDDAKGVRVRALYDYDGQEQDELSFKAGDELTKLGEEDEQGWCRGRLDSGQLGLYPANYVEAI

SEQ ID NO: 8 - protein, 23 aa, Rattus norvegicus
RRMKWKKPAGRRAPTSAPTPQRR

SEQ ID NO: 9 - protein, 23 aa, Rattus norvegicus
RRMKWKKPAGRREPTSEPTPQRR

SEQ ID NO: 10 - protein, 16 aa, Rattus norvegicus
PAGRRSPTSSPTPQRR

SEQ ID NO: 11 - protein, 30 aa, Rattus norvegicus; identical to SEQ ID NO: 6

SEQ ID NO: 12 - protein, 7 aa, Rattus norvegicus
RRMKWKK

SEQ ID NO: 13 - protein, 15 aa, Rattus norvegicus
AAVALLPAVLLALLA

SEQ ID NO: 14 - DNA, 34 bp, Rattus norvegicus
cggcgaattcaacacgaccaccgtcagcacgccc

SEQ ID NO: 15 - DNA, 34 bp, Rattus norvegicus
ctgcagaattgcggccgcttagaggtcgaagggg

SEQ ID NO: 16 - protein, 7 aa, Rattus norvegicus
RITISDP

SEQ ID NO: 17 - protein, 20 aa, Rattus norvegicus
SRSGQASPSRPESPRPPFDL

SEQ ID NO: 18 - protein, 14 aa, Rattus norvegicus
RRAPAVPPARPGSR

SEQ ID NO: 19 - protein, 8 aa, Rattus norvegicus
PAVPPARP

SEQ ID NO: 20 - protein, 4 aa, Rattus norvegicus
PARP

SEQ ID NO: 21 - protein, 25 aa, Rattus norvegicus
RRSPTSSPTPQRRAPAVPPARPGSR

SEQ ID NO: 22 - protein, 5 aa, Rattus norvegicus
SPTPQ

SEQ ID NO: 23 - protein, 19 aa, Rattus norvegicus
SPTPQRRAPAVPPARPGSR

SEQ ID NO: 24 - protein, 30 aa, Rattus norvegicus
PFGPPPQVPSRPNRAPPGVPSRSGQASPSR

SEQ ID NO: 25 - protein, 9 aa, Rattus norvegicus
GPQYGSLER

(19) United States
(12) Patent Application Publication
(10) Pub. No.: US 2008/0306007 A1
(43) Pub. Date: Dec. 11, 2008

(54) AGENTS FOR PROPHYLAXIS OR TREATMENT OF NEUROLOGICAL RELATED DISEASES AND CONDITIONS

(75) Inventors: Adam McCluskey, Mayfield (AU); Phillip Robinson, North Rocks (AU); Michael Cousin, Edinburgh (GB)

Correspondence Address: FULBRIGHT & JAWORSKI, LLP, 1301 MCKINNEY, SUITE 5100, HOUSTON, TX, 77010-3095, US

(73) Assignees: Newcastle Innovation Limited, Callaghan (AU); Children's Medical Research Institute, Weastmead (AU); The University Court of The University of Edinburgh, Edinburgh (GB)

(21) Appl. No.: 12/112,359

(22) Filed: Apr. 30, 2008

Related U.S. Application Data
(60) Provisional application No. 60/915,025, filed on Apr. 30, 2007.

(51) Int. Cl. A61K 38/10; C07K 7/08; A61P 25/00

(52) U.S. Cl. 514/14; 530/327

(57) ABSTRACT

Inhibitors of syndapin I binding to dynamin I (DynI) are provided. Examples include mimetics of a region of DynI including the serine residues S774 and S778 or phosphorylatable amino acids in homologous positions. Typically, the mimetics exclude or do not imitate at least one phosphorylation site provided by the serine residues or phosphorylatable amino acids. Peptide fragment inhibitors comprising or consisting of this region of DynI are also described. The inhibitors have application in the prophylaxis or treatment of neurological diseases or conditions. The inhibitors can also be used to inhibit neuronal cell vesicle trafficking and synaptic signal transmission.

### RELATED DISEASES AND CONDITIONS

This application claims priority to U.S. Provisional Patent Application Ser. No. 60/915,025, filed Apr. 30, 2007, which is incorporated by reference herein in its entirety.

### FIELD OF THE INVENTION

The invention relates to methods of screening for inhibitors of the interaction of syndapin I with dynamin I (DynI). The invention further relates to mimetics of a region of dynamin I (DynI), and methods for the prophylaxis or treatment of neurological related diseases or conditions.

### BACKGROUND OF THE INVENTION

Neurons communicate via the release of neurotransmitter by exocytosis from nerve terminals. After exocytosis, synaptic vesicles (SV) are retrieved by endocytosis to accommodate multiple cycles of synaptic transmission. Synaptic vesicle endocytosis (SVE) is triggered by a coordinated calcineurin-dependent dephosphorylation of a group of at least eight proteins called the dephosphins, namely DynI, amphiphysin I/II, synaptojanin, epsin, eps15, AP180 and PIP kinase Iγ (Cousin and Robinson. 2001). The dephosphins are constitutively phosphorylated in nerve terminals and their collective rephosphorylation after SVE is necessary for maintaining the continuity of SV recycling and thus maintenance of synaptic transmission. To date only one dephosphin kinase has been identified, cyclin-dependent kinase 5 (Cdk5) (Tan et al. 2003) which phosphorylates dynamin I, synaptojanin I and PIP kinase Iγ in vivo and other dephosphins such as amphiphysin I in vitro (Floyd et al. 2001). Cdk5 activity is required for SVE (Tan et al. 2003).

Dynamin I is a large GTPase enzyme, the activity of which is required for vesicle fission in SVE. The proline-rich domain (PRD) at the C-terminus contains numerous binding motifs for src-3-homology (SH3) domains, through which it interacts with proteins such as amphiphysin I, endophilin I, and syndapin I (Anggono et al. 2006). Syndapin 1 and endophilin I bind to overlapping proline rich sequences in the PRD, while amphiphysin binds at some considerable distance distal to this site (Anggono et al. 2006). The SH3-mediated dynamin I interactions of amphiphysin and endophilin have previously been reported to be involved in SVE (Gad et al. 2000) It has been proposed that different synaptic proteins like endophilin and amphiphysin are involved in mechanistically different modes of SVE, such as fast and slow modes. Amphiphysin and endophilin are able to sense membrane curvature and tubulate lipid through their Bin/Amphiphysin/RVS (BAR) domain. Syndapin I has a related F-BAR domain that can tubulate lipids (Itoh et al. 2005).

The dynamin I PRD is also the site for endogenous dynamin I phosphorylation at the synapse (Robinson et al. 1993). Cdk5 phosphorylates Ser-774 and Ser-778 in the PRD of dynamin I in vivo (Tan et al. 2003; Graham et al. 2007), but the functional role of dynamin I phosphorylation in SVE has remained obscure. It has been suggested that dynamin I dephosphorylation stimulates formation of protein complexes for endocytosis. Amphiphysin I or endophilin I have also been postulated as potential candidates for phosphorylation-dependent dynamin I binding partners (Tomizawa et al. 2003; Solomaha et al. 2005).

Most SH3 domain containing proteins bind to a PxxP core motif sequence (where x represents any amino acid) flanked by basic amino acids on one side or the other. The amphiphysin I SH3 domain binding site has been mapped exclusively to the sequence PSRPNR (SEQ ID No. 1), using dynamin I PRD deletion and site-directed mutagenesis approaches (Grabs et al. 1997) and later confirmed by surface plasmon resonance (SPR) analysis (Solomaha et al. 2005). Endophilin I has been reported to bind to a poorly defined N-terminal region of the PRD with high affinity and a C-terminal region with very low affinity (Solomaha et al. 2005). In two independent peptide scanning studies, the endophilin I SH3 domain bound two overlapping peptides SPTPQRRAPAV (Cestra et al. 1999) (SEQ ID No. 2) and RRAPAVPPARPGSRGPA (SEQ ID No. 3) (Ringstad et al. 2001) within the N-terminal half of the dynamin I PRD. The former peptide included Ser-778, but no PxxP motif, making it difficult to reconcile the studies.

### SUMMARY OF THE INVENTION

The present invention relates to the surprising finding that stimulus-dependent dynamin I dephosphorylation at S774 and S778 of the dynamin I (DynI) PRD in neurons recruits syndapin I for synaptic vesicle endocytosis (SVE) (Anggono et al. 2006), and that the inhibition of phosphorylation of one or both of these phosphorylation sites can inhibit SVE. These striking observations provide for the design and use of mimetics of a region of DynI that includes these serine residues, or phosphorylatable amino acids in homologous positions, for the treatment or prophylaxis of neurological related diseases or conditions.

In particular, the inventors have found that syndapin I binds to two adjacent regions in the dynamin I PRD (Anggono et al 2007). The first site is uniquely involved in syndapin I binding to the region DynI₇₇₂₋₇₈₄ (RRSPTSSPTPQRR) (SEQ ID No. 4). The second is a PxxP motif PAVP in DynI₇₈₆₋₇₈₉ (SEQ ID No. 5) which overlaps with the binding site for endophilin, and therefore binds both syndapin and endophilin. This duality of binding mode provides a means to specifically disrupt the dynamin I-syndapin I interaction and to block SVE by utilising a fragment of the region of DynI comprising this first site (SEQ ID No. 4) or mimetics of this region of DynI, while not affecting the dynamin I-endophilin I interaction.

In an aspect of the invention there is provided a method for prophylaxis or treatment of a neurological disease or condition in an individual, comprising administering to the individual an effective amount of an inhibitor of syndapin I binding to DynI, the inhibitor being a fragment of a region of DynI including the serine residues S774 and S778 or phosphorylatable amino acids in homologous positions, or a mimetic of the same region of DynI.

In another aspect of the invention there is provided an inhibitor of syndapin I binding to DynI, the inhibitor being a mimetic of a region of DynI including the serine residues S774 and S778 or phosphorylatable amino acids in homologous positions.

In at least one form, the mimetic will be a mimetic of the region of DynI comprising or consisting of amino acid sequence DynI₇₇₂₋₇₈₄ (RRSPTSSPTPQRR) (SEQ ID No. 4) or a homologous sequence thereof.

Typically, a mimetic embodied by the invention will exclude or not imitate at least one phosphorylation sites provided by serine residues S774 and S778 or phosphorylatable amino acids in homologous positions. For instance, the mimetic can be a mimetic in which at least one of S774 and S778, or at least one phosphorylatable amino acids in a homologous position to S774 or S778, is substituted for an amino acid other than a negatively charged amino acid. One or more further amino acid substitutions can also be made in the region of DynI including S774 and S778, or phosphorylatable amino acids in homologous positions. Respective such further substitutions will normally also comprise substitutions with other than negatively charged amino acids.

Typically, phosphorylatable amino acids in homologous positions to S774 and S778 will respectively immediately precede a proline amino acid residue. The phosphorylatable amino acids in a homologous position to S774 and S778 can be independently selected from the group consisting of serine and threonine. In an alternative form, one or both of the phosphorylatable amino acids may be tyrosine in, for example, a recombinant form of DynI.

A mimetic embodied by the invention can be inherently adapted to pass across the outer cell membrane of neurons or be coupled to a facilitator moiety for facilitating passage or translocation of the mimetic across the outer cell membrane of neurons. Generally, a mimetic embodied by the invention will be a peptide mimetic. It will also be understood the mimetic can be a mimetic of an amino acid region of DynI from a species other than human.

In another aspect, there is provided a pharmaceutical composition comprising at least one mimetic embodied by the invention together with a pharmaceutically acceptable carrier.

In another aspect of the invention there is provided a method for prophylaxis or treatment of a neurological disease or condition in an individual, comprising administering to the individual an effective amount of an inhibitor of syndapin I binding to dynamin I (Dyn I), the inhibitor being a fragment of a region of DynI including the serine residues S774 and S778 or phosphorylatable amino acids in homologous positions, or a mimetic of the region of Dyn I.

In another aspect of the invention there is provided a method for prophylaxis or treatment of a neurological disease or condition in a mammal, comprising administering to the mammal an effective amount of an inhibitor of syndapin I binding to dynamin I.

In another aspect of the invention there is provided a method for prophylaxis or treatment of epilepsy in a mammal, comprising administering to the mammal an effective amount of an inhibitor of syndapin I binding to DynI.

In another aspect of the invention there is provided a method for inhibiting synaptic signal transmission, comprising treating a neuron with an effective amount of an inhibitor of syndapin I binding to DynI.

More broadly, in another aspect of the invention there is provided a method for inhibiting synaptic vesicle endocytosis, comprising treating a neuron with an effective amount of an inhibitor of syndapin I binding to DynI.

In still another aspect of the invention there is provided the use of an inhibitor of syndapin binding to DynI in the manufacture of a medicament for prophylaxis or treatment of a neurological disease or condition in an individual.

Moreover, potential mimetics and compounds for use in methods embodied by the invention can be screened for their capacity to inhibit interaction of DynI with syndapin I and thereby SVE.

Hence, in yet another aspect there is provided a method for screening a test compound for capacity to inhibit interaction of DynI with syndapin I, comprising:

providing the test compound to be screened;

incubating the test compound with DynI or a molecule substituting for DynI in the presence of a syndapin I or a molecule substituting for syndapin I; and determining whether the compound inhibits the interaction of DynI with syndapin I.

Determination of whether the test compound inhibits the interaction of Dyn I with syndapin I can involve determining whether or not SVE is inhibited by the compound such as by methods described herein. Alternatively, the detection of inhibition of the interaction of DynI with syndapin I can be determined by immunoassays (eg., ELISA) or other appropriate assays. The test compound can for example be a small molecule, small chemical drug, a peptide, or putative peptido-mimetic or other type of mimetic.

The individual treated by a method embodied by the invention can, for instance, be a mammal such as a member of the bovine, porcine, ovine or equine families, a laboratory test animal such as a mouse, rabbit, guinea pig, a cat or dog, or a primate or human being. Typically, the mammal will be a human being.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like that has been included in this specification is solely for the purpose of providing a context for the present invention. It is not to be taken as an admission that any or all of these matters form part of the prior art base or were common general knowledge in the field relevant to the present invention as it existed anywhere before the priority date of this application.

The features and advantages of methods of the invention will become further apparent from the following detailed description of a number of embodiments.

### BRIEF DESCRIPTION OF THE ACCOMPANYING DRAWINGS

FIG. 1: Phosphorylation-dependent interaction of syndapin I and endophilin with dynamin I in vitro (a) Dynamin consists of four distinct domains: the GTP hydrolysis domain (GTPase), a pleckstrin homology (PH) domain, an assembly domain (AD) and a proline-rich domain (PRD) and is phosphorylated by Cdk5 at Ser-774 and Ser-778 in vivo in the DynI “phospho-box” (₇₆₉PAGRRSPTSSPTPQRRAPAVPPARPGSRGP₇₉₈) (SEQ ID No. 6). Point mutations were made in phospho-box residues at the indicated positions (arrows). (b) GST-DynI-PRD either WT, dmA or dmE coupled to GSH-sepharose were used in pull-downs from brain lysates. Bound proteins were separated by SDS-PAGE and stained with Coomassie Blue. (c) MALDI-MS peptide mass spectrum of a 52 kDa band identified as syndapin I. (d) MALDI-MS of the 52 kDa band revealed 42% sequence coverage of syndapin I (SEQ ID No. 7). (e) Effect of individual DynI-PRD mutants on syndapin binding in pull-down experiments (upper panels). The amount of syndapin (f) or endophilin (g) bound to GST-DynI-PRD mutants was quantified by densitometry analysis of Western blots (n=5). The data is expressed as a percent of DynI-PRD-WT ±S.E.M.

FIG. 2: Phosphorylation-dependent interaction with syndapin I in vivo. (a) Amphiphysin SH3 domain binds all synaptosomal dynamin. (b) Effect of in vivo dynamin phosphorylation on binding SH3 domains. The samples were probed with phospho-Ser-774 and phospho-Ser-778 antibodies (bottom four panels). (c) Effect of in vivo dynamin I phosphorylation on binding full length proteins. (d) Phospho-dynamin I showed reduced binding to full-length syndapin I but not full-length endophilin.

FIG. 3: Phosphopeptide mapping of dynamin I from synaptosomes. (a) Autoradiograph of a 2-D tryptic map of dynamin I from ³²Pᵢ-labelled synaptosomes. (b) Specific phosphopeptides derived from DynI₇₇₂₋₇₈₄ (RRSPTSSPTPQRR) (SEQ ID No. 4 that accounted for each spot of panel (a) were identified by MALDI-TOF MS. (c) Graph showing phospho-Ser-774 predominates 2:1.

FIG. 4: Depolarization-regulated interaction of syndapin I with dynamin I in synaptosomes. (a) A dynamin I-syndapin I complex in synaptosomes. Blots are representative of at least 3 independent experiments. (b) Depolarisation stimulates the formation of a calcineurin-sensitive dynamin I-syndapin I complex. (c) The phospho-box peptide releases syndapin I from the complex. Blots are representative of two independent experiments.

FIG. 5: Dynamin I phospho-box peptide inhibits SVE in synaptosomes. (a) Sequences of penetratin-linked peptides (penetratin tag—DynI₇₆₉₋₇₈₄ AA (SEQ ID No. 8) and penetratin tag—DynI₇₆₉₋₇₈₄ EE (SEQ ID No. 9)) based on DynI₇₆₉₋₇₈₄ peptide (SEQ ID No. 10) comprising the phospho-box sequence. (b) The AA mutant blocks SV turnover. (c) Ca²⁺-dependent glutamate release from control (Ctrl) or peptide-treated synaptosomes stimulated with KCl (solid bar) is unaffected by either peptide. (d) Both peptides have small background effects on SV exocytosis. (e) SVE is specifically inhibited by the AA peptide. Retrieval efficiency (SV turnover/exocytosis) is displayed using exocytosis data from either panel c or d. (f) Hypotonically lysed synaptosomes loaded with FM2-10 (g) Ca²⁺-dependent glutamate release from control (Ctrl) or peptide-treated synaptosomes stimulated with 4-AP (1 mM, solid bar), n=3.

FIG. 6: DynI^{dmA} and Dyn^{dmE} inhibit SVE in cerebellar granule neurons. (a) Primary cultures of CGNs were transfected with DynI^{WT}-GFP (green) and stimulated uptake of FM4-64 (red) was visualized by fluorescence microscopy. (b) Monochrome FM4-64 image shows puncta representing sites of SV turnover. (c) CGNs were depolarized to unload the accumulated FM4-64. The fluorescence of all puncta was reduced. After transfection of DynI^{dmA}-GFP (d-f) or Dyn^{dmE}-GFP (g-i) into CGNs there was considerably less accumulation of FM4-64 (e and h). Scale bar is 5 μm. (j) The extent of FM4-64 accumulation and thus SV turnover. Solid bars indicate the period of stimulation. (k) Both Ser-774 and Ser-778 contribute to SV turnover in an additive fashion (l) SV exocytosis is unaffected. Exocytosis was determined by examining the kinetics of FM4-64 unloading.

FIG. 7: Overexpression of DynI^{dmA} and Dyn^{dmE} arrest SVE when assayed using synaptopHluorin. CGN cultures were co-transfected with synaptopHluorin and either DynI^{WT}-mCerulean (a), DynI^{dmE}-mCerulean (b) or DynI^{dmA}-mCerulean (c). (d) Collated data showing the amount of fluorescence remaining (i.e. synaptopHlourin remaining on the cell surface) 60 s after termination of KCl stimulation (n=27 DynI^{WT}-mCerulean; n=34 Dyn^{dmE}-mCerulean and n=12 DynI^{dmA}-mCerulean ±s.e.m.).

FIG. 8: A schematic diagram for the mapping of syndapin I binding region (SEQ ID No. 11) within the dynamin I PRD (SEQ ID No. 6) is shown. Two SH3-domain binding motifs (PxxP) immediately after the phosphorylation sites are identified as site 2 and site 3. Grb2 and amphiphysin I binding sites at the carboxy-terminus are identified site 8 and site 9 respectively. Point mutations were made in the various PxxP motif residues at the indicated positions (arrow).

FIG. 9: A schematic diagram showing DynI-PRD mutants of the site 2 and site 3 PxxP motifs in the DynI PRD domain (DynI₇₆₉₋₈₄₁). Only partial sequences DynI₇₆₉₋₇₉₈ (SEQ ID No. 6) and (DynI₈₂₇₋₈₄₁) are shown.

FIG. 10: A schematic diagram illustrating the binding orientation of syndapin I and endophilin I within the DynI PRD domain (SEQ ID No. 6) is shown. The basic residues flanking the site 2 and site 3 PxxP motifs of the Dyn I PRD domain were mutated to either glutamic acid or alanine residues.

FIG. 11: Graph showing the Dyn I phosphobox peptides DynI₇₆₉₋₇₈₄AA and DynI₇₆₉₋₇₈₄EE do not block internalization of Alexa-labelled Tfn in non-neuronal U2OS cells.

### DETAILED DESCRIPTION OF EMBODIMENTS OF THE INVENTION

S774 and/or S778 of DynI (or at least one phosphorylatable amino acid in a homologous position to either of these serine residues) can be replaced by amino acids other than a negatively charged amino acid in a mimetic peptide embodied by the invention. The DynI amino acid sequence can be that of any eukaryotic species including rodent (such as rat), primate, human and porcine DynI sequence. A mimetic can also have one or more other amino acid differences compared to the native DynI amino acid sequence. Such additional amino acid changes can comprise the addition, deletion and/or substitution of one or more amino acids. Inversion of amino acids and other mutational changes that result in modification of the DynI amino acid sequence are also encompassed. Moreover, a mimetic peptide of the invention can comprise an amino acid or amino acids not encoded by the genetic code (e.g., a synthetic or other non-naturally occurring amino acid). For example, D-amino acids rather than L-amino acids can be utilized to inhibit endopeptidase degradation of the mimetic in vivo. The term “peptide” is used herein interchangeably with “polypeptide”. Moreover, it will be understood a native peptide fragment of DynI comprising or consisting of amino acid sequence DynI₇₇₂₋₇₈₄ (RRSPTSSPTPQRR) (SEQ ID No. 4) or a homologous sequence thereto can be utilized in a method embodied by the invention, and the invention expressly encompasses such native peptide sequences per se.

The substitution of an amino acid can be a conservative or non-conservative substitution. The term conservative amino acid substitution is to be taken in the normally accepted sense of replacing an amino acid residue with another amino acid having similar properties which substantially does not adversely affect the capacity of the mimetic peptide to inhibit interaction of DynI with syndapin I. For example, a conservative amino acid substitution can involve substitution of a basic amino acid such as arginine with another basic amino acid such as lysine. Likewise, for instance, a non-polar amino acid may be substituted with another non-polar amino acid such as alanine.

In one or more forms, a mimetic embodied by the invention will typically have basic amino acids in positions corresponding to R769 and R770 and also basic amino acids in positions corresponding to R783 and R784. In at least some mimetics, at least one of R769 and R770 and at least one of R783 and R784 of DynI will be retained in the mimetic in homologous positions. Moreover, in particular, one or more of amino acids of amino acid sequence DynI₇₇₂₋₇₈₄ (RRSPTSSPTPQRR) (SEQ ID No. 4) such as T776, S777, T780 and/or Q782 may respectively be substituted for an amino acid other than an acidic amino acid such as aspartic acid (D) or glutamic acid (E). Generally, a mimetic as described herein will have proline residues (P) in homologous positions to DynI₇₇₂₋₇₈₄ (RRSPTSSPTPQRR) (SEQ ID No. 4), although mimetics in which one or more of the prolines of SEQ ID No. 4 are respectively substituted for an amino acid other than an acidic amino acid residue may be provided.

Typically, a mimetic peptide embodied by the invention will have amino acid sequence identity with the region of DynI including S774 and S778 of about 50% or greater, and more usually about 60%, 70%, 80% or 90% or greater, and all sequence homologies and ranges thereof within those enumerated above are expressly encompassed. Sequence identity between amino acid sequences is determined by comparing amino acids at each position in the sequences when optimally aligned for the purpose of comparison. The sequences are considered identical at a position if the amino acids at that position are the same.

The peptide mimetic can be up to about 40 amino acids in length or greater. Usually, the peptide will have a length of less than 40 amino acids, and generally about 35, 30, 25, 20, or 15 amino acids in length or less. Typically, the peptide will have a length of greater than about 10 amino acids and more usually, a length in a range of from about 13 to 25 amino acids and more usually, from about 13 to about 20 amino acids. It will also be understood that all individual peptide lengths and ranges of peptide lengths falling within the various ranges enumerated above (eg., 14, 15, 16, 17, 18 or 19 amino acids in length etc.) are expressly encompassed.

A peptide mimetic can be prepared by introducing nucleotide change(s) in a DynI nucleic acid sequence such that the desired amino acid changes are achieved upon expression of the nucleic acid in a host cell or for instance, by chemically synthesising the peptide or the nucleic acid encoding the peptide. The provision and use of fusion proteins incorporating a peptide mimetic as described herein is also expressly provided for by the invention. Nucleic acid encoding a fusion protein can be provided by joining separate DNA fragments encoding the peptide mimetic and a carrier peptide as describe further below for facilitating passage of the mimetic into a target cell by employing blunt-ended termini and oligonucleotide linkers, digestion to provide staggered termini and ligation of cohesive ends as required. Recombinant techniques for providing peptide mimetics and fusion proteins embodied by the invention are well known to the skilled addressee (eg., see also Ausubel et al. (1994) Current Protocols in Molecular Biology, USA, Vol. 1 and 2, John Wiley & Sons, 1992, Sambrook et al (1998) Molecular cloning: A Laboratory Manual, Second Ed., Cold Spring Harbour Laboratory Press, New York, and subsequent editions and updates of the foregoing).

Peptide mimetics and fusion proteins described herein can be expressed in vitro and purified from cell culture for administration to an individual in accordance with the invention. The nucleic acid will generally first be introduced into a cloning vector and amplified in host cells, prior to the nucleic acid being excised and incorporated into a suitable expression vector for transfection of cells. Typical cloning vectors incorporate an origin of replication (ori) for permitting efficient replication of the vector, a reporter or marker gene for enabling selection of host cells transformed with the vector, and restriction enzyme cleavage sites for facilitating the insertion and subsequent excision of the nucleic acid sequence of interest. Preferably, the cloning vector has a polylinker sequence incorporating an array of restriction sites. The marker gene may be drug-resistance gene (eg., Amp^{r} for ampicillin resistance), a gene encoding an enzyme such as chloramphenicol acetyltransferase (CAT), β-lactamase, adenosine deaminase (ADA), aminoglycoside phosphotransferase (APH), dihydrofolate reductase (DHFR), hygromycin-B-phosphotransferase (HPH), thymidine kinase (TK), or for instance β-galactosidase encoded by the E. coli lacZ gene (LacZ′). Yeast reporter genes include imidazole glycerolphosphate dehydratase (HIS3), N-(5′-phosphoribosyl)-anthranilate isomerase (TRP1) and β-isopropylmalate dehydrogenase (LEU2). As will be appreciated, expression vectors of the invention can also incorporate such marker genes.

Cloning vectors that can be used include cloning vectors for mammalian, yeast and insect cells. Particular vectors that may find application include pBR322 based vectors and pUC vectors such as pUC118 and pUC119. Suitable expression vectors include plasmids and cosmids capable of expression of a DNA (eg., genomic DNA or cDNA) insert. An expression vector will typically include transcriptional regulatory control sequences to which the inserted nucleic acid sequence is operably linked. By “operably linked” is meant the nucleic acid insert is linked to the transcriptional regulatory control sequences for permitting transcription of the inserted sequence without a shift in the reading frame of the insert. Such transcriptional regulatory control sequences include promoters for facilitating binding of RNA polymerase to initiate transcription, expression control elements for enabling binding of ribosomes to transcribed mRNA, and enhancers for modulating promoter activity. The expression vector employed will depend on the host cell to be transfected, the mode of transfection and the required level of transcription of the nucleic acid insert.

Numerous expression vectors suitable for transfection of prokaryotic (eg., bacterial) or eukaryotic (e.g., yeast, insect or mammalian cells) are known in the art. Expression vectors suitable for transfection of eukaryotic cells include pSV2neo, pEF.PGK.puro, pTk2, pRc/CNV, pcDNAI/neo, adenoviral vectors and pAdEasy based expression vectors most preferably incorporating a cytomegalovirus (CMV) promoter. For expression in insect cells, baculovirus expression vectors can be utilised examples of which include pVL based vectors such as pVL1392, and pVL941, and pAcUW based vectors such as pAcUW1. Any means for achieving the introduction of nucleic acid into cells for expression of the encoded peptide or fusion protein can be used. Transfer methods known in the art include viral and non-viral transfer methods. Suitable virus into which appropriate viral expression vectors may be packaged for delivery to target cells include adenovirus, vaccinia virus, retroviruses of avian, murine and human origin, herpes viruses including Herpes Simplex Virus (HSV) and EBV, papovaviruses such as SV40, and adeno-associated virus. Particularly preferred virus are replication deficient recombinant adenovirus. Suitable expression and cloning vectors are for instance described in Molecular Cloning. A Laboratory Manual., Sambrook et al., 2nd Ed. Cold Spring Harbour Laboratory., 1989, and subsequent editions thereof.

Rather than utilising viral mediated transfection of cells, mimetics, expression vectors for expression of peptide mimetics and the like can be introduced into a cell in vitro or in vivo by liposome mediated transfection. The liposomes can carry targeting or binding molecules for maximising efficiency of delivery to the target cells. Such targeting molecules include antibodies or binding fragments thereof (eg., Fab and (Fab′)₂), ligands and cell surface receptors for facilitating fusion of liposomes to the target cells of interest. Alternatively, nucleic acid sequences and the like may be intracellularly delivered in vitro using conventional cold or heat shock techniques or for instance, calcium phosphate coprecipitation or electroporation protocols as are known in the art. Yet another strategy is to design the mimetic or fusion protein to have the inherent ability to pass across the lipid bilayer of a cell such as by the use of amino acid sequences rich in hydrophobic amino acid residues.

A particularly preferred way of achieving intracellular delivery of a mimetic embodied by the invention is to use a carrier peptide or other suitable such facilitator moiety for facilitating passage of the mimetic across the outer cell membrane. Carrier peptides known in the art include penetratin and variants thereof, human immunodeficiency virus Tat derived peptide, transportan derived peptide, signal peptide, and partial sequences of the foregoing such as the penetratin fragment RRMKWKK (SEQ ID No. 12). Signal peptides that may be used for delivery of a peptide mimetic embodied by the invention into cells as described herein include signal peptide for Kaposi fibroblast growth factor (e.g., see U.S. Pat. No. 5,807,746), and partial sequences thereof comprising or incorporating the amino acid sequence AAVALLPAVLLALLA (SEQ ID No. 13). Rather than a carrier peptide, the facilitator moiety for facilitating transport or translocation of the peptide mimetic, fusion protein or other mimetic active into the cell can be a lipid moiety, fatty acid or other non-peptide moiety which enhances cell membrane permeability of the mimetic. However, any molecule that can affect passage of the mimetic across the outer cell membrane and which is physiologically acceptable can be used. The mimetic can be coupled to the selected facilitator moiety by an amino acid linker sequence, by a peptide bond or non-peptide covalent bond, a cross-linking agent, or for example, by charge association between the mimetic and the facilitator moiety. Chemical ligation methods can also be used to create a chemical bond between the carboxy terminal amino acid of a signal peptide, penetratin sequence or the like and the mimetic.

Host cells that can be used for expression of peptide mimetics or fusion proteins in accordance with embodiments of the invention include bacteria such as E. coli, Bacillus strains (eg., B. subtilis), Streptomyces and Pseudomonas bacterial strains, yeast such as Sacchromyces and Pichia, insect cells, avian cells and mammalian cells such as Chinese Hamster Ovary cells (CHO), COS, HeLa, HaRas, W138, SW480, and NIH3T3 cells. The host cells are cultured in a suitable culture medium under conditions for expression of the introduced nucleic acid prior to purification of the expressed product from the host cells, and/or supernatants as the case may be using standard purification techniques well known in the art.

Peptide mimetics, fusion proteins and the like embodied by the invention can also be modified by coupling one or more additional moieties to the peptide to improve solubility, lipophilic characteristics to enhance uptake by cells, stability, biological half-life, or for instance to act as a label for subsequent detection or the like. Such modifications can result from post-translational or post-synthesis modification such as by the attachment of carbohydrate moieties, or chemical reaction(s) resulting in structural modification(s) (eg., the alkylation or acetylation of one or more amino acid residues or other changes involving the formation of chemical bonds).

Further mimetics for inhibiting the interaction of DynI and syndapin I can also be provided based on peptide mimetics as described above. Such further mimetics can be non-peptide mimetics or, for example, comprise a proteinaceous or peptide moiety coupled to a non-peptide moiety (peptido-mimetics). The design and provision of such further mimetics can involve determining the physical properties of the region of DynI comprising S774 and S778 (or phosphorylatable amino acids in homologous positions) such as size and charge distribution, and the tertiary structure of the region including flanking amino acids. Moreover, the DynI amino acid sequence can be modeled taking into account the stereochemistry and its conformation as determined using x-ray crystallography, nuclear magnetic resonance and/or commercially available computer modelling software. Such modelling techniques are well known in the art. The provision of a mimetic can for example involve selecting or deriving a template molecule onto which chemical groups are attached to provide physical and chemical characteristics of the mimetic or for further chemical reactions for achieving the required physical and chemical characteristics. The selection of template molecules and chemical groups is based on ease of synthesis, pharmacological acceptability, potential for degradation in vivo, stability and maintenance of biological activity upon administration, and outer cell membrane solubility for passage into target cells. Accordingly, a mimetic can be a small molecule (e.g., a small chemical entity) modeled on DynI₇₇₂₋₇₈₄ (RRSPTSSPTPQRR) (SEQ ID No. 4) or a homologous amino acid sequence thereof.

A mimetic as described herein can also be constrained in a conformation required for activity, synthesised with side chain structures or with cysteine residues for forming a disulfide bridge to improve stability or to enhance activity, or otherwise be incorporated into a molecule with a known stable structure in vivo. A peptide or other mimetic for use in a method embodied by the invention may also be cyclised to provide enhanced rigidity and thereby stability in vivo. Various methods for cyclising peptides, fusion proteins or the like are known to the skilled addressee. For example, a synthetic peptide incorporating two cysteine residues distanced from each other along the peptide can be cyclised by the oxidation of the thiol groups of the residues to form a disulfide bridge between them. Cyclisation can also be achieved by the formation of a peptide bond between the N-terminal and C-terminal amino acids of a peptide or for instance, through the formation of a bond between the positively charged amino group on the side chain of a lysine residue and the negatively charged carboxyl group on the side chain of a glutamic acid residue. Variation of cycle size for optimisation of activity can be achieved by synthesising peptides in which the position of amino acids for achieving cyclisation has been altered. The formation of direct chemical bonds between amino acids or the use of any suitable linker to achieve a desired three-dimensional conformation is also well within the scope of persons skilled in the art.

Any suitable assay protocol can be employed in a method for screening proposed mimetics and test compounds for capacity to inhibit the interaction of DynI with syndapin I or more generally synaptic vesicle endocytosis, including competitive and non-competitive assays. The test compound can be any type of molecule, e.g., including non-peptide moieties such as small chemical molecules/entities, whether or not it is considered a putative inhibitor of the binding of syndapin I to DynI. Suitable immunoassays which can be used include antibody capture and enzyme linked immunosorbent assays (ELISA). Such assays include those in which the DynI-syndapin I complex is detected by direct binding with a labelled antibody, and those in which the DynI-syndapin I complex is captured by a first antibody, typically immobilized on a solid substrate (e.g., a microtitre tissue culture plate formed from a suitable plastics material such as polystyrene or the like), and the complex is then treated with labelled second antibody specific to DynI or syndapin I wherein the binding of the second antibody is detected by a signal emitted by the label. Such sandwich techniques are well known. Antibodies used in assays embodied by the invention can be polyclonal or monoclonal antibodies, and can be bound to a solid substrate covalently utilising any commonly used amide or ester linkers, or by adsorption.

Rather than full length DynI, a fragment such as the DynI-PRD domain or partial sequence thereof or for example, a mutant form of DynI, can be used in a screening assay embodied by the invention. The mutant form can, for instance, be a truncated form of DynI or modified form of the protein with one or more amino acid changes compared to wild-type DynI. However, it will be understood any such molecule which can substitute for wild type or full length DynI for the purpose of the assay can be utilised. Similarly, fragments, full length, truncated or mutant forms of syndapin I can also be utilised in a screening assay embodied by the invention.

The “label” used in an assay as described herein can be any molecule which by its nature is capable of providing or causing the production of an analytically identifiable signal which allows the detection of the antibody complex. Such detection may be qualitative or quantitative. An antibody can, for instance, be labelled with a radioisotope such as ³²P, ¹²⁵I or ¹³¹I, an enzyme, a fluorescent label, chemiluminescent molecule or an affinity label such as biotin, avidin, streptavidin and the like. An enzyme can be conjugated with an antibody by means of coupling agents such as glutaraldehyde, carbodiimides, or for instance, periodate although a wide variety of conjugation techniques exists. Commonly used enzymes include horseradish peroxidase, glucose oxidase, β-galactosidase and alkaline phosphatase amongst others.

Substrates for enzyme based detection systems will generally be chosen for production upon hydrolysis of a detectable colour change. However, fluorogenic substrates can also be used which yield a fluorescent product rather than a chromogen. Suitable fluorescent labels include those capable of being conjugated to an antibody substantially without altering the binding capacity of the antibody, examples of which include fluorescein, phycoerythrin (PE) and rhodamine which emit light at a characteristic wavelength in the colour range following illumination with light at a different wavelength.

Methods for labelling antibodies can be found in, for example, Current Protocols in Molecular Biology, Ausubel F M., John Wiley & Sons Inc. Enzyme based assay protocols suitable for use in methods embodied by the invention are also described for instance in Handbook of Experimental Immunology, Weir et al., Vol. 1-4, Blackwell Scientific Publications 4^{th} Edition, 1986 and subsequent editions thereof. Optimal concentrations of antibodies, temperatures, incubation times and other assay conditions can be determined by reference to conventional assay methodology and the application of routine experimentation.

Endocytosis is a major contributor or direct cause of diverse human diseases. A list of vesicle trafficking-specific diseases has been published, see for example Aridor and Hannan 2000, Traffic 1:836-851 and Aridor and Hannan 2002, 3:781-790 the contents of which are incorporated herein by reference in their entirety. In addition, for example, growth factor receptors (e.g. EGF-R) require dynamin for internalisation and maintenance of cellular activities from signalling to cell growth (Seto et al. 2002). Dynamin is central to all endocytic trafficking from the cell surface, the Golgi apparatus, endosomes and mitochondria. Several neurodegenerative diseases are associated with these trafficking pathways and the endocytotic pathway has been implicated in the generation of β-amyloid (Aridor & Hannan 2000). Diseases and conditions in which endocytosis plays a role in the brain include Alzheimer's disease, Huntington's disease (HD), stiff-person syndrome, Lewy body dimentias, and Niemann-Pick type C disease (Cateldo et al. 2001; Metzler et al. 2001; Ong et al. 2001; Smith et al. 2000).

In Alzheimer's disease (AD), β-amyloid precursor protein (APP) is internalized from axonal cell surfaces in clathrin-coated vesicles and sorted away from recycling synaptic vesicles, where it is transported to endosomes and the cell soma (Marquez-Sterling et al. 1997). The endosome is the first compartment along the dynamin-dependent endocytic pathway after internalization of APP or ApoE (Smythies J. 2000) and endosomal alterations are evident in pyramidal neurons in Alzheimer brain (Cataldo et al. 1997). Endocytic pathway activation is prominent in APP processing and β-amyloid formation and is an early feature of neurons in vulnerable regions of the brain in sporadic Alzheimer's disease (Cataldo et al. 2001).

Huntington's disease (HD) is a neurodegenerative disorder principally affecting striatal neurons, yet the mutated gene product huntingtin is not brain-specific. Huntingtin interacts strongly with members of the Huntingtin-interacting protein 1 (HIP 1) family. The huntingtin-HIP1 interaction is restricted to the brain and is inversely correlated to the polyglutamine length in huntingtin. Loss of normal huntingtin-HIP1 interaction may contribute to a defect in membrane-cytoskeletal integrity in the brain. HIP1 is a fundamental component of the dynamin-mediated endocytic machinery (Metzler et al. 2001). Numerous reports have linked the neurological defects in HD to endocytosis abnormalities (Aridor & Hannan, 2000; Metzler. 2001).).

Another example is the presynaptic synuclein protein associated with contributing to Lewy body diseases, including Parkinson's disease, Lewy body dementia and a Lewy body variant of AD. Exogenous synuclein causes neuronal cell death due to its endocytosis and formation of intracytoplasmic inclusions. Cell death and α-synuclein aggregates are direct consequences of its endocytosis in human neuroblastoma cells (Sung et al. 2001). In the brain, further neurological diseases and conditions in which endocytosis plays a role include stiff person syndrome, and Niemann-Pick type C disease. Endocytosis is also implicated in epilepsy. For example, mice with targeted disruption of either of the two endocytic proteins synaptojanin (SJ) and amphiphysin have reduced SVE and die from random seizures throughout their lives (Di Paolo et al. 2002) showing a role for SVE in neuronal excitability and a direct link to epilepsy.

Endocytic pathways are also utilized by viruses, toxins and symbiotic microorganisms to gain entry into cells. For instance, botulism neurotoxins and tetanus neurotoxin are bacterial proteins that inhibit transmitter release at distinct synapses and cause two severe neuroparalytic diseases, tetanus and botulism. Their action is dependent on their internalisation via endocytosis into nerve terminals (Humeau et al. 2000). Hence targeting endocytosis and particularly SVE with inhibitors has application as a clinically useful strategy. It follows from the above that neurological diseases or conditions which may be treated in accordance with one or more embodiments of the invention include diseases and conditions responsive to inhibition of DynI, diseases and conditions associated with cell vesicle trafficking, diseases and conditions associated with synaptic signal transmission, neurodegenerative diseases, neuropathic pain, psychotic and psychiatric conditions, epilepsy, β-amyloid associated diseases, Alzheimer's disease, Parkinson's disease, Huntington's disease, stiff person syndrome and Lewis body dementias, neuroparalytic diseases, and Niemann-Pick type C disease amongst others.

Neuropathic pain typically develops when peripheral nerves are damaged through surgery (including spine surgery), bone compression in cancer, diabetes, infection (including shingles or HIV infection), AIDS, alcoholism, amputation, chemotherapy, facial nerve problems, or multiple sclerosis, and is a major factor causing impaired quality of life for millions of people worldwide (Scadding. 2003). Anti-convulsant drugs such as Phenyloin and Gabapentin are highly efficacious in treating neuropathic pain (Wiffen et al. 2000; Scadding. 2003). These drugs act through modulation of synaptic vesicle transmission, indicating the potential of the modulation of synaptic vesicle endocytosis in treating this debilitating condition. That is, inhibition of the interaction of syndapin I with DynI which halts synaptic vesicle endocytosis modulates synaptic vesicle transmission, may halt or limit pain signalling.

Mimetics and fragments of DynI as described herein can be administered to an individual in need of such treatment alone or be co-administered with one or more other therapeutic compounds or drugs. For example, a mimetic peptide can be co-administered in combination with drugs conventionally used for treating neurological diseases and disorders. By “co-administered” is meant simultaneous administration in the same formulation or in two different formulations by the same or different routes, or sequential administration by the same or different routes wherein the mimetic peptide and other therapeutic compound(s) or drug(s) exert their physiological effect during overlapping periods.

The fragment of DynI/mimetic can be also be designed or otherwise adapted to enhance in vivo stability and/or efficacy of the peptide as described above. For example, a mimetic can also be pegylated, orthinylated, or provided in the form a dendrimer to decrease the rate of clearance from the bloodstream via the kidneys, to increase resistance to protease cleavage (e.g., by endopeptidases) and/or to enhance passage of the mimetic into cells. Pegylation of peptides for instance is well known to the skilled addressee. For instance, a mimetic as described herein may be coupled to 2 or more monomers of polyethylene glycol (PEG) and more usually, from about 4 to about 10 monomers of PEG. When provided in a dendrimer form, any suitable dendrimer framework may be utilised. Peptide dendrimers are particularly suitable for use in methods of the invention. Peptide dendrimers in at least some embodiments of the invention comprise monomers of the mimetic coupled to a branched framework of polyamino acids (typically lysine branching units). The dendrimer will typically have at least 3 layers/generations of amino acid branching units, the monomers of the mimetic being coupled to the outermost layer/generation of the amino acid branching units such that the dendrimer presents at least 4 separate monomer units of the mimetic. The mimetic can be grafted onto the surface of the outermost layer/generation of polyamino acid branching units forming the framework of the dendrimer, or be synthetically assembled on the polyamino acid branching units of the dendrimer.

Dendrimer frameworks are also commercially available, and are typically formed from organic amino compounds such as poly (amidoamine) (PAMAM), tris(ethylene amine) ammonia or poly (propylene imine) (Astramol™) to which separately prepared mimetics as described herein can be covalently linked. Suitable dendrimer framework to which a mimetic as described herein can be coupled, and methods for the provision of dendrimers including peptide dendrimers are, for example, described in Lee et al, 2005; Sadler and Tam, 2002; and Cloninger, 2002, the entire contents of which are incorporated herein in their entirety by reference. As will be understood, dendrimers incorporating fragments of DynI as described herein can also be provided.

The fragment(s) of Dyn I/mimetic(s) will generally be formulated into a pharmaceutical composition comprising the fragment(s)/mimetic(s) and a pharmaceutically acceptable carrier. Pharmaceutical compositions include sterile aqueous solutions suitable for injection and sterile powders for the extemporaneous preparation of injectable solutions. Such injectable compositions will be fluid to the extent that syringability exists. Injectable solutions will typically be prepared by incorporating the active(s) in the selected carrier prior to sterilising the solution by filtration. In the case of sterile powders, preferred methods of preparation are vacuum drying and freeze-drying techniques which yield a powder of the active and any additional desired ingredient from previously sterile filtered solutions thereof.

For oral administration, the fragment/mimetic may be formulated into any orally acceptable carrier deemed suitable. In particular, the fragment/mimetic can be formulated with an inert diluent, an assimilable edible carrier or it may be enclosed in a hard or soft shell gelatin capsule. Moreover, a fragment/mimetic can be provided in the form of ingestable tablets, buccal tablets, troches, capsules, elixirs, suspensions or syrups. Fragments of DynI/mimetics as described herein can also be formulated into topically acceptable preparations including creams, lotions or ointments for internal or external application. Topically acceptable compositions can be applied directly to the site of treatment including by way of dressings and the like impregnated with the preparation.

A pharmaceutical composition of the invention can also incorporate one or more preservatives such as parabens, chlorobutanol, phenol, and sorbic acid. In addition, prolonged absorption of the composition may be brought about by the inclusion of agents for delaying absorption such as aluminium monosterate. Tablets, troches, pills, capsules and like can also contain one or more of the following: a binder such as gum tragacanth, acacia, corn starch or gelatin; a disintegrating agent such as corn starch, potato starch or alginic acid; a lubricant such as magnesium sterate; a sweetening agent such as sucrose, lactose or saccharin; and a flavouring agent.

Pharmaceutically acceptable carriers include any suitable conventionally known physiologically acceptable solvents, dispersion media, isotonic preparations and solutions including for instance, physiological saline. Use of such ingredients and media for pharmaceutically active substances is well known. Except insofar as any conventional media or agent is incompatible with the fragment of DynI/mimetic, use thereof is expressly encompassed. It is particularly preferred to formulate compositions in dosage unit form for ease of administration and uniformity of dosage. Dosage unit form as used herein is to be taken to mean physically discrete units, each containing a predetermined quantity of the active calculated to produce a therapeutic or prophylactic effect. When the dosage unit form is a capsule, it can contain the active in a liquid carrier. Various other ingredients may be present as coatings or to otherwise modify the physical form of the dosage unit. For instance, tablets, pills or capsules may be coated with shellac, sugars or both.

The pharmaceutical compositions embodied by the invention will generally contain at least about 0.1% by weight of the fragment of DynI/mimetic up to about 80% w/w of the composition. The amount in the composition will be such that a suitable effective dosage will be delivered to the individual taking into account the proposed mode of administration. Preferred oral compositions will contain between about 0.1 μg and 4000 mg of the fragment/mimetic.

The dosage of the fragment of DynI/mimetic embodied by the invention will depend on a number of factors including whether the active is to be administered for prophylactic or therapeutic use, the disease or condition for which the active is intended to be administered, the severity of the condition, the age of the individual, and related factors including weight and general health of the individual as may be determined in accordance with accepted medical principles. For instance, a low dosage may initially be given which is subsequently increased at each administration following evaluation of the individual's response. Similarly, frequency of administration can be determined in the same way that is, by continuously monitoring the individual's response between each dosage and if necessary, increasing the frequency of administration or alternatively, reducing the frequency of administration.

Typically, the fragment of DynI/mimetic will be administered in accordance with a method embodied by the invention at a dosage up to about 50 mg/kg body weight and preferably, in a range of from about 5 μg/kg to about 100 μg/kg body weight.

Routes of administration include but are not limited to respiritoraly, intravenously, intraperitonealy, subcutaneously, intramuscularly, by infusion, orally, rectally, topically and by implant. With respect to intravenous routes, particularly suitable routes are via injection into blood vessels which supply the target tissue to be treated. The fragment(s)/mimetic(s) can also be delivered into cavities such for example the pleural or peritoneal cavity, or be injected directly into tumour tissue. Suitable pharmaceutically acceptable carriers and formulations useful in compositions of the present invention may for instance be found in handbooks and texts well known to the skilled addressee, such as “Remington: The Science and Practice of Pharmacy (Mack Publishing Co., 1995)”, the contents of which is incorporated herein in its entirety by reference.

The present invention will be described herein after with reference to a number of non-limiting Examples.

### EXAMPLE 1

### Identification of Syndapin I Binding to DynI

### 1. Materials and Methods

### 1.1.1 DNA Constructs

Dynamin I-Green fluorescent protein (rat sequence, Iaa isoform) in pEGFP-N1 was provided by Dr Mark A. McNiven (Mayo Clinic, Minnesota) (Cao et al. 1998). The sequence encoding the dynamin Iaa-PRD (rat, amino acids 746-864) was amplified from this GFP-tagged dynamin Iaa with the oligonucleotides 5′-CGGCGAATTC-AACACGACCACCGTCAGCACGCCC-3′ (SEQ ID No. 14) and 5′-CTGCAGAATT-GCGGCCGCTTAGAGGTCGAAGGGG-3′ (SEQ ID No. 15) and then subcloned into pGEX4T-1 vector (Amersham Biosciences) as previously described (Anggono et al. 2006). Underlining indicates unique restriction sites used for subcloning the amplified cDNA. Dynamin I point mutants were generated using the QuickChange site-directed mutagenesis kit (Stratagene) and were confirmed by DNA sequencing. All GST-fusion proteins were expressed in Escherichia coli and purified using glutathione (GSH)-sepharose beads (Amersham Biosciences) according to the manufacturer's instructions. The sequence of the rat dynamin Iaa used in the present study is a composite of Genbank Accession No. P21575 (also NP_{—}542420) rat D100 with the last 5 amino acids of the tail (RITISDP) (SEQ ID No. 16) being replaced by the amino acid sequence SRSGQASPSRPESPRPPFDL (SEQ ID No. 17 (Genbank Accession No. S36762). (www.ncbi.nim.gov/Genbank/index.html. National Centre for Biotechnology Information, US National Library of Medicine, Rockville Pike, Bethesda, Md., United States).

### 1.1.2 Pull-Down Studies

Total rat brain extract was prepared by homogenising brain tissue in ice-cold lysis buffer (1% Triton X-100, 150 mM NaCl, 25 mM Tris pH 7.4, 1 mM EDTA, 1 mM EGTA, 20 μg/ml leupeptin, 1 mM phenylmethylsulfonyl fluoride and EDTA-free Complete protease inhibitor (Roche)). The homogenate was centrifuged twice at 75,600 g for 30 min at 4° C. The supernatant was pre-cleared by addition of GSH-sepharose beads for 1 h, pelleted at 50 g for 5 min at 4° C., and the supernatant collected. GST-DynI-PRD recombinant proteins were then incubated with an equal amount of tissue lysate at 4° C. for 1 h. Beads were washed extensively with ice-cold 20 mM Tris pH 7.4 containing 1 mM EGTA, eluted in 2×SDS-PAGE sample buffer, resolved on 7.5-15% gradient SDS gels and stained with colloidal Coomassie Blue. Identification of proteins was by matrix-assisted laser desorption ionization time-of-flight mass spectrometry (MALDI-TOF MS). Some peptides were sequenced by tandem MS/MS.

### 1.1.3 Synaptosomes and ³²Pᵢ Labelling

Crude (P2) synaptosomes were prepared from rat brain and labelled with ³²Pᵢ as described previously (Robinson et al. 1993). Synaptosomes were lysed in ice-cold lysis buffer and centrifuged at 20,442 g for 20 min at 4° C. Most pull-down studies using synaptosomes were performed sequentially. First, dynamin I was isolated from the supernatant for 1 h at 4° C. using GST-syndapin I, GST-endophilin I or GST-amphiphysin I, either full-length recombinant proteins or their SH3 domains alone, bound to GSH-sepharose. Secondly, GST-AmphI-SH3 domain was used in a subsequent pull-down study to recover any dynamin I not captured in the first pull-down. The washed beads were heated in SDS-PAGE sample buffer and proteins were resolved on SDS gels and subjected to autoradiography.

### 1.1.4 Glutamate Release Assay

In all SV recycling studies synaptosomes were prepared from rat cerebral cortex by centrifugation on discontinuous percoll gradients (Dunkley et al. 1986). The glutamate release assay was performed as described before (Cousin and Robinson 2000a). Data is presented as Ca²⁺-dependent glutamate release, calculated as the difference between release in plus and minus Ca²⁺ solutions. Penetratin peptides (Genemed Synthesis) were preincubated with the synaptosomes for 30 min before stimulation and had no effect on Ca²⁺-independent release of glutamate.

### 1.1.5 SV Turnover and Internalisation Assays

Loading of FM2-10 (Molecular Probes) into recycling synaptosomal SVs was measured as previously described. Data is presented as Ca²⁺-dependent FM2-10 unloading, which is the difference between release after loading in plus and minus Ca²⁺ solutions. Where indicated, synaptosomes were preincubated with penetratin peptide for 30 min prior to stimulation of FM2-10 loading. Neither peptide affected the ability of the standard pulse of KCl to evoke release of FM2-10-labelled vesicles, since parallel studies on S2 Ca²⁺-dependent glutamate release showed no effect. The same assay was used to monitor the effect of penetratin peptide on SV exocytosis. However, in this instance, the synaptosomes were loaded with FM2-10 in plus Ca²⁺ Krebs-like solution and, where indicated, were preincubated with penetratin peptide for 30 min prior to stimulation of FM2-10 unloading. Data is presented as Ca²⁺-dependent FM2-10 unloading. The SV internalisation assay was performed as previously described.

### 1.1.6 Cell Culture

Primary cerebellar granule neuron (CGN) cultures were prepared from 7-day old Sprague-Dawley rat pups as previously described (Tan et al. 2003). In brief, neurons were plated on poly-D-lysine coated glass coverslips at a density of 1.5×10⁵ cells/coverslip. Neurons were cultured in MEM containing Earle's salts plus 10% (v/v) foetal calf serum, 25 mM KCl, 30 mM glucose, 2 mM glutamine, 100 U/ml penicillin and 100 μg/ml streptomycin at 37° C., in a humidified atmosphere of 5% CO₂. The culture medium was supplemented with 10 μM cytosine arabinoside after 24 hours. Transfections were carried out using an established calcium phosphate precipitation protocol. CGNs were transiently transfected with 1.5 μg of DNA between 7-10 days in vitro and were used 48 h later.

### 1.1.7 Fluorescence Imaging and Image Analysis

The effect of overexpression of dynamin I-GFP constructs on SV recycling in CGNs was monitored using the styryl dye FM4-64 (Molecular Probes). The extent of FM4-64 loading was quantified by normalising the fluorescence value at the start of stimulation to an arbitrary value and monitoring the total decrease on unloading with a dual KCl stimulus. The rate of FM4-64 unloading (as a measure of SV exocytosis) was estimated by determining the time taken for individual nerve terminals to lose 50% of their dye content on stimulation of unloading. These values were normalised to untransfected nerve terminals within the same field of view. SV exocytosis and recycling was also monitored using the pH-sensitive VAMP-GFP (synaptopHluorin). Dynamin constructs linked to mCerulean rather than to GFP were used in this part of the study. CGNs were co-transfected with synaptopHluorin and either DynI^{WT}-mCerulean, DynI^{dmA}-mCerulean or Dyn^{dmE}-mCerulean and stimulated with 50 mM KCl for 20 sec. The evoked synaptopHluorin response was recorded at 500 nm excitation and 510-550 nm emission (mCerulean fluorescence was not detectable at this wavelength). Co-expression of dynamin I was confirmed by recording at 430 nm excitation. Responses were analysed by calculating the increased fluorescence of synaptopHluorin over baseline for individual nerve terminals and then normalising the maxima and minima to 1 and 0.

### 1.2 Results

### 1.2.1 Dynamin I Phosphorylation-Dependent Protein Partners

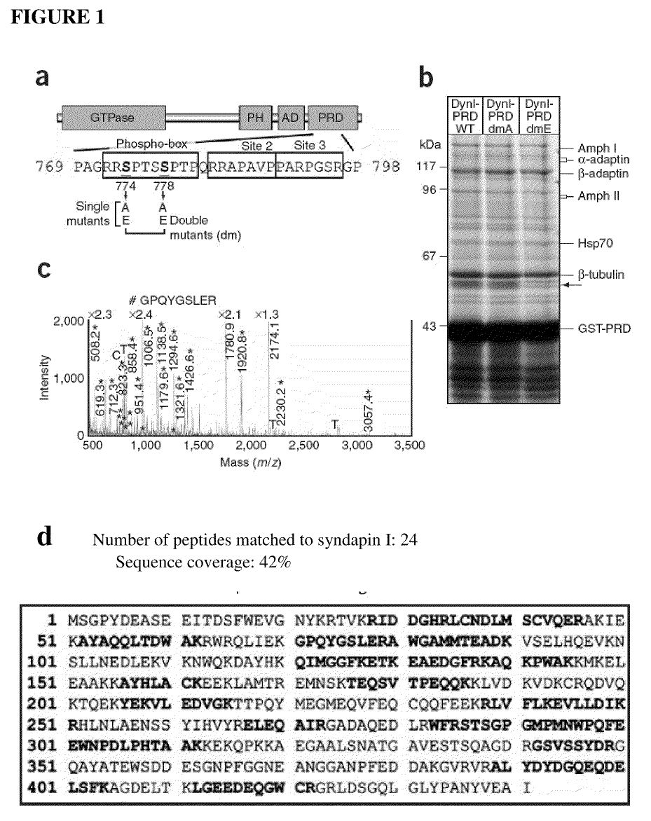

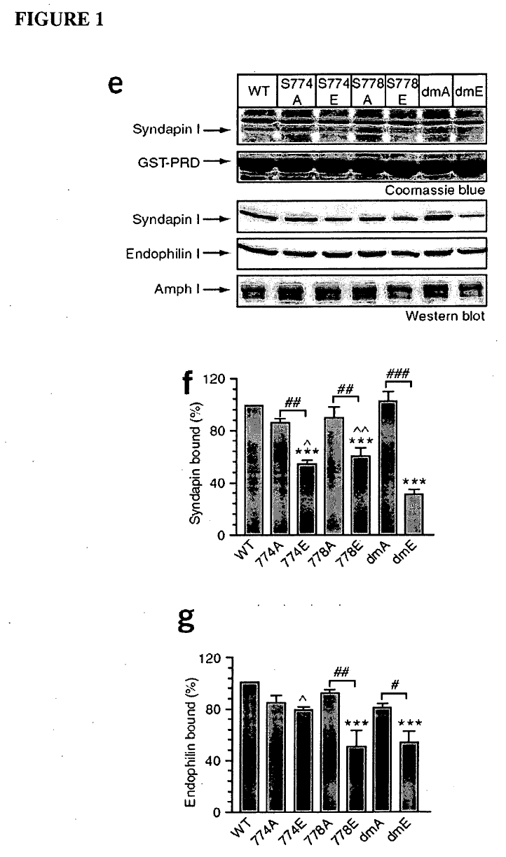

Dynamin I is phosphorylated in synaptosomes on Ser-774 and Ser-778 and a previous report of phosphorylation at Thr-780 (Tomizawa et al. 2003) is an in vitro artifact (Graham et al 2007). To identify proteins whose interaction with dynamin I might be regulated by these sites a series of single or double point mutations was generated (FIG. 1a). Ser to Ala mutation prevents phosphorylation while Ser to Glu mutation should mimic phosphorylation. Dynamin I PRD, either WT, dmA (non-phosphorylatable) or dmE (pseudo-phosphorylation) were expressed as GST-fusion proteins. A GST pull-down screen for binding partners revealed several known partners such as amphiphysin I/II and their binding partners, α-adaptin and β-adaptin (see FIG. 1b), each being identified using MALDI-TOF MS (not shown). Pull-downs with WT and dmA or dmE DynI-PRDs were essentially the same, except the binding of one protein of 52 kDa was specifically inhibited in the DynI-PRD-dmE pull-down (FIG. 1b, arrow). MALDI-MS peptide mass spectra of the 52 kDa band identified it as syndapin I (also known as PACSIN I). A representative MALDI-TOF MS peptide mass map is shown (FIG. 1c). The intensity of the 4 largest peaks were reduced by the indicated amounts for display purposes (X2.3 etc.) It revealed 24 matching peptides with 42% sequence coverage of syndapin I (FIG. 1d). The tryptic peptide detected at m/z of 1,006.50 was sequenced by tandem MS/MS and matched 100% to the GPQYGSLER (SEQ ID No. 25) sequence of syndapin I. The interaction of the other dynamin I binding proteins was not significantly regulated by pseudo-phosphorylation and therefore, their dynamin binding was independent of syndapin. In particular, the binding of amphiphysin I or II was barely affected (FIG. 1b). This suggests Ser-774 and Ser-778 phosphorylation inhibits the interaction of dynamin I with syndapin I, but not with amphiphysin I/II.

To determine individual roles for the two phospho-sites, DynI-PRD pull-downs with Ser-774 or Ser-778 individually mutated to Ala or Glu were performed. Syndapin I binding was decreased with either of the pseudo-phosphorylated point mutants, but not the Ala mutations (FIG. 1e, top panel). The effect of the two sites was almost additive. These results were confirmed by Western blot analysis (FIG. 1e, bottom panel) and by quantitative densitometry analysis (FIG. 1f). Hence, both sites are involved in syndapin I binding, while the two sites act coordinately for maximal effect.

Another major dynamin interacting protein is endophilin I, the SH3 domain of which shows reduced binding to Cdk5-phosphorylated dynamin I PRD in vitro (Solomaha et al. 2005). Since endophilin I was not detected by Coomassie Blue stain, migrating close to the GST-PRD, the pull-downs were probed with anti-endophilin I antibodies (Santa Cruz, Calif.). Endophilin I binding was significantly reduced by the S778E mutation, but not by the S774E mutation (FIG. 1e, bottom panel, and FIG. 1g). It also showed reduced binding to DynI-PRD-dmE to the same extent as S778E alone. Amphiphysin I binding was unaffected by any of the mutations (FIG. 1e). Therefore, syndapin I and endophilin I are potential phosphorylation-dependent binding partners for dynamin I. However, syndapin I binding is regulated by both sites, while endophilin I is regulated by only a single site, Ser-778.

### 1.2.2 a Phosphorylation-Regulated Dynamin I-Syndapin I Complex

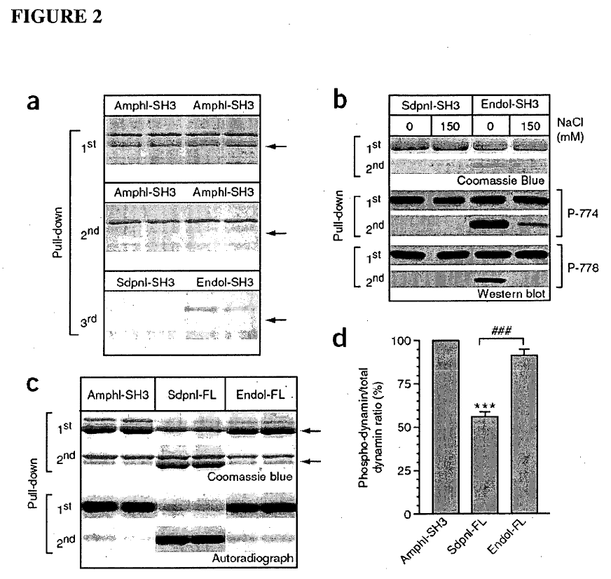

The preceding studies utilised a recombinant dynamin I domain to identify full-length native binding partners in tissues. To confirm biological function of the full-length dynamin I protein, reverse pull-down experiments using recombinant syndapin I or endophilin I to capture native dynamin I with its in vivo phosphorylation status preserved were performed. The method employed sequential pull-downs, based on observations that GST-AmphI-SH3 quantitatively binds synaptosomal dynamin independently of its phosphorylation status (Tan et al. 2003) (FIG. 2a-c).

A pull-down with AmphI-SH3 recovered considerable dynamin I protein (FIG. 2a, top panel in quadruplicate, while a second pull-down with AmphI-SH3 recovered less than 1% residual dynamin (provided the molar ratio of GST-AmphI-SH3:dynamin exceeded 2:1; FIG. 2a, middle panel). A third sequential pull-down with two different SH3 domains recovered no residual dynamin protein (FIG. 2a, bottom panel), nor was anything detectable by Western blots with dynamin I or phosphorylation site-specific antibodies (not shown). The band migrating above dynamin was a bacterial contaminant of the recombinant protein expression. All panels were from the same gel. The results show that AmphI-SH3 domain binds essentially all dynamin I regardless of its in vivo phosphorylation status and can be used to capture residual dynamin.

The ability of isolated SH3 domains of syndapin I or endophilin I to bind endogenous dynamin I from lysed synaptosomes, which contain a large pool of phosphorylated dynamin I, was then examined after 45 min incubation in a Krebs-like buffer at 37° C. was then examined. The remaining lysate was used for a second pull-down with GST-AmphI-SH3. Surprisingly, recombinant GST-SH3 domains gave different results to DynI-PRD. GST-SdpnI-SH3 bound all detectable dynamin I, regardless of its in vivo phosphorylation status (FIG. 2b, upper panels). The presence of the endogenously phosphorylated form of dynamin I was confirmed by blots with the two phosphorylation site-specific antibodies (FIG. 2b, lower four panels). EndoI-SH3 extracted at least 95% of the synaptosomal dynamin I in the absence of salt and the residual unbound dynamin I was captured in the second pull-down by AmphI-SH3. The unbound dynamin I was highly phosphorylated, indicating that the interaction of endophilin I with dynamin I has a phosphorylation-regulated component. However, when pull-downs were performed in the absence of salt (conditions not reflecting the ionic strength inside cells) there can be considerable non-specific binding. Indeed, the presence of 150 mM salt strengthened EndoI-SH3 binding to phospho-dynamin I, and virtually no phosphorylation-dependent interaction was evident (FIG. 2b). Thus full-length syndapin I and endophilin I have significant differences in their binding properties to dynamin I in comparison to their SH3 domains alone.

Since the interaction of the isolated SH3 domains did not match those observed using pull-downs with dynamin I PRD, the question arose as to whether the association of dynamin I with full-length syndapin I or endophilin I might be phosphorylation-regulated. Studies with ³²Pᵢ-labelled synaptosomes were then performed to reveal the endogenous phosphorylation status of dynamin I by autoradiography. ³²Pᵢ-labelled synaptosomal lysates were incubated with the full-length GST-fusion proteins of syndapin I (SdpnI-FL) or endophilin I (EndoI-FL) in the presence of 150 mM NaCl, followed by a second pull-down with GST-AmphI-SH3. While AmphI-SH3 domain bound almost all dynamin I and all the phosphorylated form in the first pull-down (FIG. 2c, top panels), SdpnI-FL showed considerably reduced interaction with dynamin I. SdpnI-FL bound almost exclusively to unphosphorylated dynamin I (FIG. 2c, bottom panels). In contrast, EndoI-FL strongly bound almost all dynamin I, independently of its phosphorylation status. Quantitative analysis showed that full-length syndapin I had significantly reduced binding to phospho-dynamin I relative to the total dynamin I bound to the beads (56±3% of AmphI-SH3 control), while endophilin I binding to dynamin I was clearly independent of its phosphorylation status (91±4% of AmphI-SH3 control). Hence, it was concluded that syndapin I is the relevant dynamin I phosphorylation-dependent protein partner for dynamin I in synaptosomes.

As syndapin I and endophilin I utilise distinct subsets of phosphorylation sites for in vitro binding, a study was undertaken to further discriminate between their in vivo roles. It was surmised that if the distribution of in vivo phosphate between Ser-774 and Ser-778 favoured the former, then syndapin I binding may be more relevant. To determine if either of the two phosphorylation sites might predominate in synaptosomes, in vivo phosphorylated dynamin I was digested with trypsin and subjected to 2-D tryptic peptide mapping (Graham et al. 2007).

Six related tryptic phosphopeptides migrated as a group of spots (FIG. 3a) that completely account for the total number of theoretical tryptic peptides that could be produced from digestion of DynI₇₇₂₋₇₈₄ (RRSPTSSPTPQRR) (SEQ ID No. 4) (which includes the phospho-box). MALDI-TOF MS and tandem MS/MS were used to identify and directly sequence the phosphopeptide within each spot (FIG. 3b, the peptides phosphorylated on both Ser-774 and Ser-778 are in brackets). No phosphorylation on Thr was found, again suggesting that a previous report on Thr-780 was an artifact of in vitro phosphorylation studies (Tomizawa et al. 2003). Spots A-C were derived from dynamin phosphorylated on a single site, at least 99% of which was Ser-774, while spots D-F were from the doubly phosphorylated form. Summing the total ³²Pᵢ incorporation revealed a ratio of 3:1 for doubly (774+778) versus singly (774 alone) phosphorylated dynamin I. Due to the presence of two Arg residues at each end of the phospho-box, peptides B and E have the Arg at either end (shown with a dashed line) and each presents in two forms which are chemically identical. The phosphopeptides were identified as the following dynamin I sequences with phosphorylation at Ser-774 (A-C) or Ser-774+Ser-778 (D-F): A 774-783+80, m/z=1,137.6; B 773-783+80 and/or 774-784+80, m/z=1,293.6; C 773-784+80, m/z=1,449.8; D 774-783+160, m/z=1,217.5; E 773-783+160 and/or 774-784+160, m/z=1,373.7; E 773-784+160, m/z=1,529.6. Phospho-Ser-774 was found to predominate 2:1 over Ser-778 (FIG. 3c). Therefore, phospho-Ser-774 is the major phosphate present on synaptosomal dynamin I and phospho-Ser-778 rarely exists in isolation, supporting the conclusion that the phosphorylation-regulated dynamin I binding partner is syndapin I rather than endophilin I.

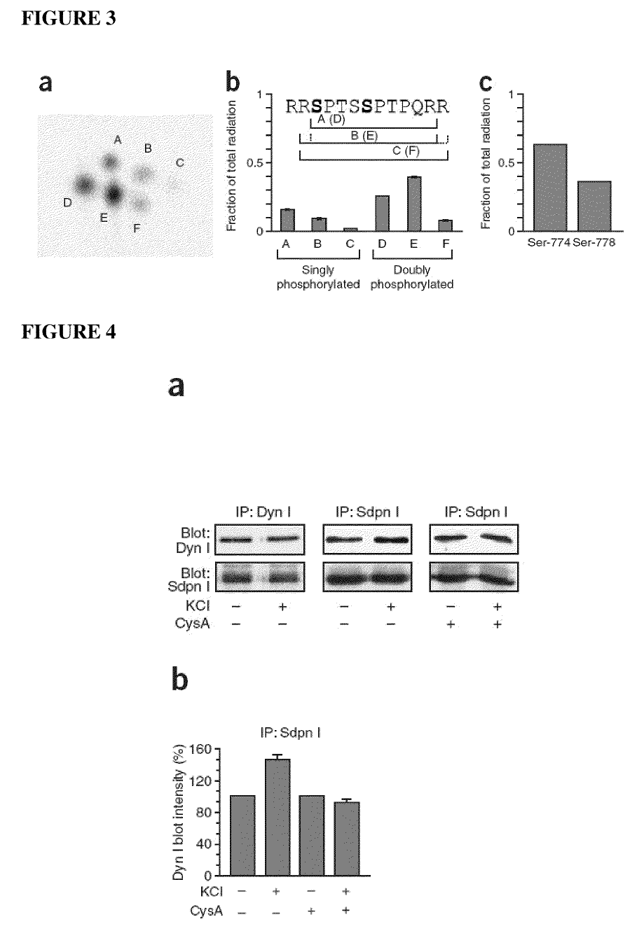

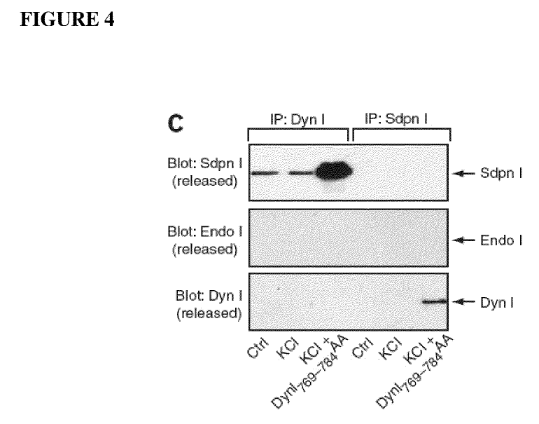

Next, the question was asked whether the dynamin I-syndapin I interaction is regulated by nerve terminal stimulation which activates calcineurin-dependent dynamin I dephosphorylation. Dynamin I and syndapin I were immunoprecipitated from either resting or KCl depolarised synaptosomes. Briefly, synaptosomes were preincubated in the presence or absence of 40 μM cyclosporin A (CysA), stimulated for 5 s (30 mM KCl), lysed, co-immunoprecipitated (Co-IP) then were blotted for dynamin I and syndapin I. The two proteins co-immunoprecipitated, thereby demonstrating that their interaction occurs in vivo, with depolarization causing dephosphorylation of dynamin I and stimulating the formation of calcineurin sensitive dynamin I-syndapin I complex (FIG. 4a-b). This suggests that a specific endocytic protein complex is formed during SVE. The amount of dynamin I immunoprecipitated by the syndapin I antibody (as in panel a) was quantified using densitometry (n=3, data are mean ±s.e.m.). To confirm the role of calcineurin, synaptosomes were treated with the antagonist cyclosporin A and the increased association of syndapin I with dynamin I was abolished.

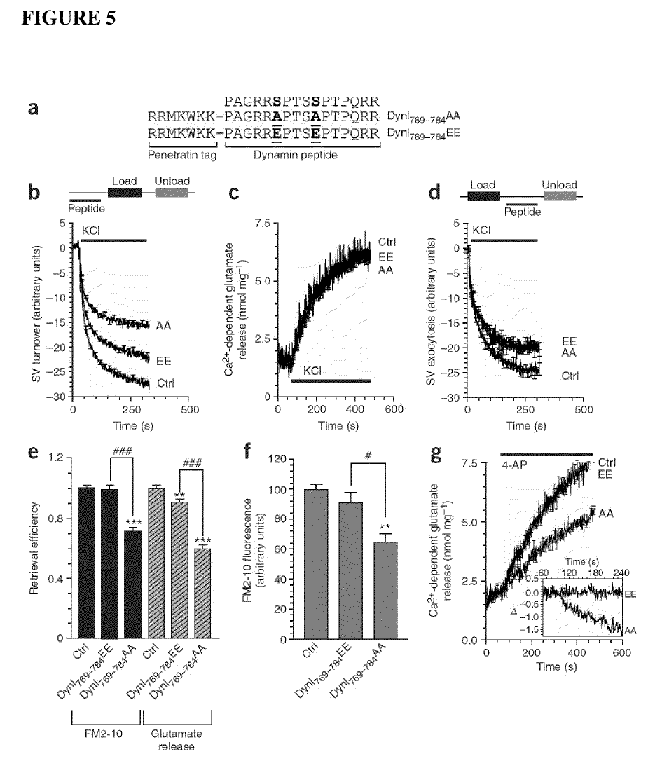

To determine whether the phospho-box sequence might account for syndapin I recruitment to dynamin I, the ability of a DynI₇₆₉₋₇₈₄AA peptide (FIG. 5a) to disrupt their interaction was tested. Syndapin I or dynamin I were immunoprecipitated from nerve terminals and the individual protein complexes were incubated with DynI₇₆₉₋₇₈₄AA (SEQ ID No. 8) peptide (see below). Their interaction in both cases was reduced by the peptide, as indicated by the specific release of either binding partner from the bead-bound complex (FIG. 4c). No elution of endophilin I (FIG. 4c) or amphiphysin I (data not shown) was detected, highlighting the specificity of the interaction. Thus, the dynamin I phospho-box sequence can compete for syndapin I binding within the endocytic complex, but not endophilin I, demonstrating the central role of the phospho-box region in mediating complex formation.

### 1.2.3 SVE is Inhibited by Dynamin I Phospho-Box Peptides

SVE in synaptosomes was measured to determine whether the phosphorylation sites in dynamin I play a direct role in SVE. Since isolated nerve terminals cannot be transfected with DNA, a membrane-permeable peptide strategy was employed. Mimetic peptides (DynI₇₆₉₋₇₈₄AA (SEQ ID No. 8) and DynI₇₆₉₋₇₈₄EE (SEQ ID No. 9)) were designed corresponding to the amino acid sequence of the dynamin I phospho-box (FIG. 5a) and were tagged with a penetratin heptapeptide RRMKWKK (SEQ ID No. 12) to facilitate their delivery into synaptosomes. Briefly, synaptosomes were preincubated in the presence or absence of either penetratin peptide (250 μM) prior to FM2-10 loading stimulated by 30 mM KCl. Accumulated FM2-10 was unloaded by another KCl stimulus (indicated by the solid bar). SV turnover is displayed for control (Ctrl) or peptide treated synaptosomes. The DynI₇₆₉₋₇₈₄AA peptide specifically reduced the dynamin I-syndapin I interaction, but not the dynamin I-endophilin I interaction (FIG. 4c). If the phosphorylation-regulated syndapin I interaction itself is essential for SVE, the penetratin-linked peptides may inhibit the process. Both peptides significantly reduced SV turnover to differing extents (FIG. 5b). The DynI₇₆₉₋₇₈₄AA peptide (250 μM) inhibited turnover by more than 40% (57.1±2.1% of control), compared to less than 20% with the DynI₇₆₉₋₇₈₄EE peptide (250 μM, 81.1±2.4% of control).

A block in SV turnover cannot be ascribed to an inhibition of SVE until an effect on exocytosis has been excluded. Two independent exocytosis assays were conducted to assess this: endogenous glutamate release and FM2-10 unloading. Neither the DynI₇₆₉₋₇₈₄AA nor DynI₇₆₉₋₇₈₄EE peptide had any significant effect on KCl-evoked Ca²⁺-dependent glutamate release (DynI₇₆₉₋₇₈₄AA −92.5±8.4% of control; DynI₇₆₉₋₇₈₄EE −90.2±9.5% of control, FIG. 5c). Both peptides had small effects on FM2-10 unloading evoked by 30 mM KCl (DynI₇₆₉₋₇₈₄AA −81.1±0.8% of control; DynI₇₆₉₋₇₈₄EE −82.3±0.6% of control, FIG. 5d). Finally, the extent of SVE inhibition by the peptides was quantified by calculating “retrieval efficiency” which takes into account prior exocytosis; a value of less than one indicating a selective SVE inhibition. DynI₇₆₉₋₇₈₄EE had little effect on retrieval efficiency using data from either exocytosis assay, whereas there was a robust inhibition with DynI₇₆₉₋₇₈₄AA (FIG. 5e). Thus DynI₇₆₉₋₇₈₄AA exerts a selective inhibition of SVE in synaptosomes (FIG. 5e) by disrupting the dynamin I-syndapin I interaction (FIG. 4c).

To directly demonstrate that the block of SVE by DynI₇₆₉₋₇₈₄AA was due to an inhibition of SV retrieval from the plasma membrane, rather than a post-endocytic recycling defect, an FM2-10 SV internalisation assay was performed, based on purification of SVs after their loading and directly quantifying their dye content. If SVE was arrested before SV fission occurred, a reduction in FM2-10 content of the SVs should be apparent, while a block of recycling should produce no such reduction. SVs were purified and their fluorescence values were corrected for dye uptake in the absence of Ca²⁺ (Ca²⁺-dependent) and normalised to control (n=3, mean ±s.e.m.). DynI₇₆₉₋₇₈₄AA inhibited FM2-10 accumulation by approximately 35% (64.9±5.5% of control), whereas DynI₇₆₉₋₇₈₄EE had no significant effect (90.7±7.1% of control, FIG. 5f).

Selective inhibition of SVE by the phospho-box peptide should produce a rundown or fatigue of glutamate release after repeated stimuli, since the supply of SVs will progressively decline. To produce repetitive stimulation in synaptosomes the K⁺ channel blocker 4-aminopyridine (4-AP) was used. This produces a tetrodotoxin-sensitive repetitive firing that mimics in vivo stimulation. It has previously been reported that 4-AP evokes continual SV recycling for at least 15 minutes in synaptosomes. The DynI₇₆₉₋₇₈₄AA peptide produced an activity-dependent depression of release that increased with continued stimulation (FIG. 5g). Significantly, it was without effect on the first 45 sec of 4-AP evoked exocytosis (see inset in FIG. 5g), emphasising the activity-dependent nature of the rundown. The inset shows the subtracted value for the AA or EE peptides from the control exocytosis (A). The inset is also zoomed on the first 3 min from 4-AP addition (which was at 60 s) to highlight the lack of effect during the first 45s. The results show that blocking syndapin I binding to dynamin I produces a functional block in SVE that induces synaptic fatigue.

### 1.2.4 Dynamin I Phosphorylation Sites are Required for SVE

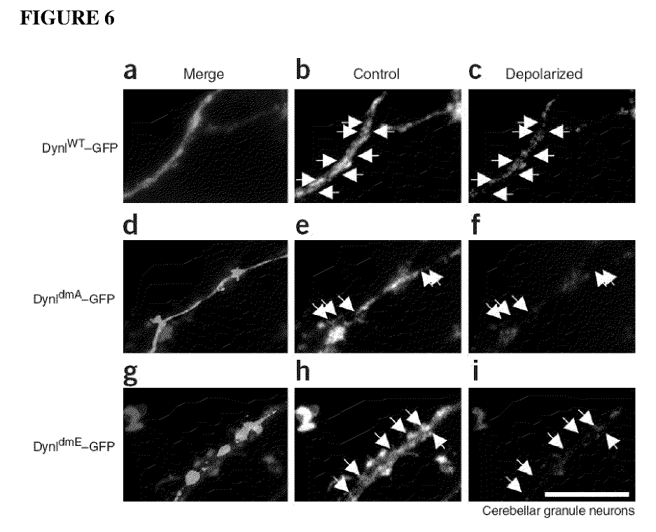

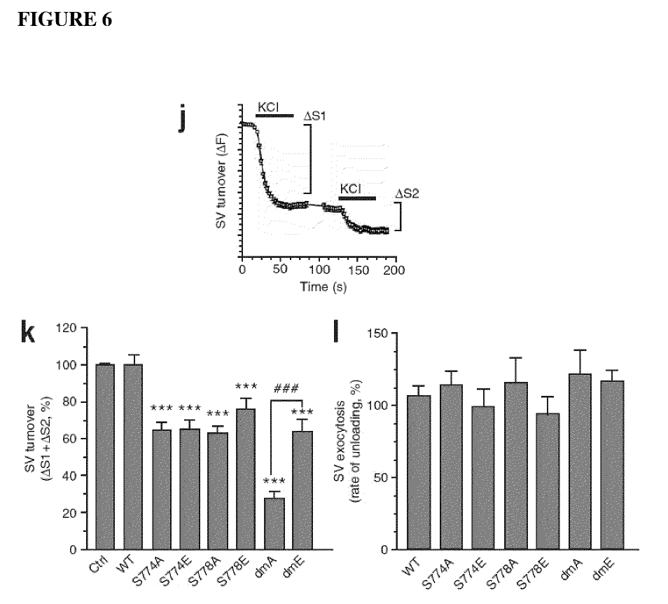

To show the key role of the phosphorylation sites by independent methods in cultured neurons their role in SV turnover in nerve terminals of CGNs transfected with dynamin I-GFP was examined. SV turnover in neurons transfected with DynI^{WT}-GFP was indistinguishable from that in untransfected neurons (FIG. 6a-c). However, the double mutants Dyn^{dmA}-GFP (FIG. 6d-f) or DynI^{dmE}-GFP (FIG. 6g-i) produced greatly reduced loading of FM4-64 (Molecular Probes, Eugene Oreg.). The extent of HM4-64 accumulation and thus SV turnover, was quantified from the change in fluorescence (AF) by unloading the CGN terminals with two sequential KCl stimuli (S1 and S2) (FIG. 6j). The total SV recycling pool was reduced by approximately 70% in CGNs transfected with DynI^{dmA}-GFP (28±3.5% of control) and 40% with DynI^{dmE}-GFP (64.4±6.2% of control) (FIG. 6k). However, transfection of DynI^{WT}-GFP had no detectable effect (100.1±5.2% of control, FIG. 6k).

The phosphorylation sites were then examined separately. Both Ala and Glu mutation on Ser-774 and Ser-778 reduced SV turnover by approximately 35% each (FIG. 6k). However, the inhibitory effect of DynI^{dmA}-GFP was even greater, being additive with that of the two single mutants. This shows that both sites contribute to SV turnover. The reduction in the total SV recycling pool could be the result of a decrease in either SVE or exocytosis. To discriminate between these two possibilities, the kinetics of FM4-64 unloading after stimulation with KCl was examined, since this is independent of the extent of dye accumulation. The unloading kinetics were not significantly different with any construct (FIG. 6l). Thus, the reduction in the total SV recycling pool was mediated by an inhibition of SVE and not by an effect on exocytosis.

To address whether the inhibition of FM4-64 uptake was due to a SVE arrest or to an impaired rate of SVE, FM4-64 was applied to transfected sparse cultures of CGNs after stimulation. Sparse cultures avoided potential artefacts from intertwining of transfected and non-transfected neurons. There was no increase in dye accumulation after transfection with DynI^{dmA}-GFP or DynI^{dmA}-GFP relative to DynI^{WT}-GFP, indicating SVE arrest.

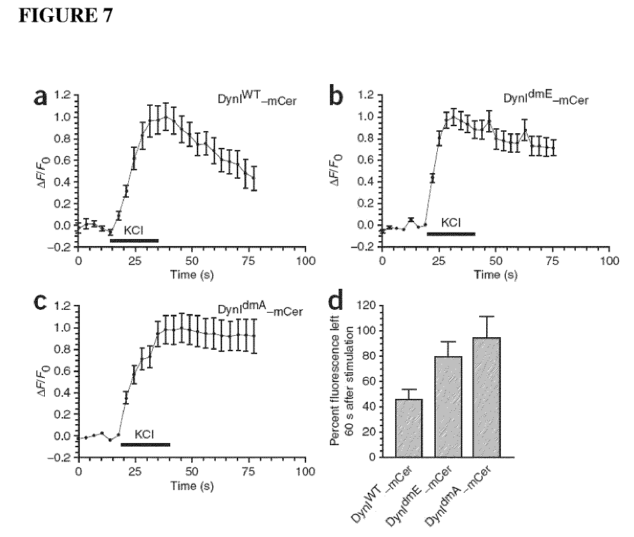

An SVE assay that was independent of FM dyes was then used to determine the significance of the dynamin I phosphorylation sites. SynaptopHluorin is a fusion construct of a pH-sensitive GFP with the integral synaptic vesicle protein synaptobrevin/VAMP, that detects vesicle fusion by an increased fluorescence and vesicle retrieval by a subsequent decrease. When neurons were cotransfected with synaptopHluorin and DynI^{WT}-mCerulean, KCl evoked robust exocytosis, followed by SVE on stimulus removal (FIG. 7a). DynI^{dmA}-mCerulean and Dyn^{dmE}-mCerulean did not affect exocytosis, but each selectively inhibited SVE (FIG. 7b-c). Representative time courses of synaptopHluorin fluorescence responses evoked by 50 mM KCl are displayed, with fluorescence normalised to a maximum of 1 unit. The change in fluorescence (AF) normalized to initial fluorescence (F₀), averaged over the first six data points, is plotted as a function of time. The bar indicates the 20 s period of KCl addition. These results provide further biological evidence that the S774 and S778 phospho-sites of DynI specifically regulate neuronal SVE.

### 1.3 Discussion

Calcineurin stimulates SVE through dephosphorylation of the dephosphins. Dynamin I dephosphorylation has previously been suggested to regulate protein complex formation with amphiphysin I or endophilin I. However, the present study has demonstrated these effects do not occur either in vivo or with the full-length proteins. The results further reveal that the biological significance and functional role of dynamin I phosphorylation on Ser-774 and Ser-778 is to control syndapin I recruitment for SVE. Dynamin I and syndapin I form a complex in nerve terminals, their interaction is increased by rapid depolarization and this can be reversed by treatment with calcineurin antagonists. This places syndapin I at the centre of the main endocytic machinery for SVE at the synapse.

A number of major physiological correlations were observed supporting the conclusion that the dynamin-syndapin interaction is essential for SVE, and thereby synaptic transmission. Firstly, the DynI₇₆₉₋₇₈₄AA phospho-box peptide blocked syndapin I binding, not endophilin I, reducing its availability for recruitment by endogenous dynamin I for SVE in synaptosomes. When introduced into synaptosomes, this correlated with SVE inhibition. Secondly, as expected for a specific endocytic block, the DynI₇₆₉₋₇₈₄AA peptide also produced a progressive rundown of glutamate release after prolonged repetitive stimulation induced by 4-AP (FIG. 5g) but not KCl (FIG. 5c). This emphasises the central role of SVE in sustaining synaptic transmission. The apparent conflict between results with 4-AP and KCl is not surprising and is due to the stimulation method. Prolonged KCl stimulation does not support efficient SV recycling at or after 300 s of stimulation in synaptosomes (Cousin and Robinson 2000b), potentially due to desensitization of Ca²⁺-channels or an increased prevalence of bulk endocytosis. Thirdly, when transfected into neurons, DynI^{dmA}-GFP blocked SVE in two independent types of assay—uptake of FM4-64 or retrieval of membrane-inserted synaptopHluorin. Finally, both Ser-774 and Ser-778 contributed individually to syndapin I binding, their effects being additive. When DynI^{S774A}-GFP and DynI^{S778A}-GFP single mutants were transfected into neurons, the resultant inhibition of SVE precisely correlated with the in vitro effects of each mutant. This reveals an essential biological role for both Ser-774 and Ser-778 in SVE and suggests that the two sites operate coordinately to control both SVE and syndapin I binding. This is the first demonstration that mutation of a phosphorylation site in an endocytic protein creates a dominant-negative function in SVE. Finally, both the Ala and Glu mutations interfere with the cycle of dynamin I dephosphorylation and phosphorylation by preventing appropriate binding and release (respectively) of syndapin.

The results establish syndapin I functions as a component of a major endocytic protein complex important immediately before dynamin I recruitment to the recycling SV. In this model, the F-BAR domain of syndapin I induces plasma membrane curvature and/or shapes the neck of the budding vesicle, but has no ability to cut the neck. For vesicle fission, it appears dynamin I is dephosphorylated upon depolarisation, binds the SH3 domain of syndapin I, co-encircling the vesicle neck and provides energy from GTP hydrolysis for SV fission.

### EXAMPLE 2

### Identification of the DynI Binding Sites for Syndapin I and Endophilin I

### 2.1 Materials and Methods

Peptides were synthesised by Mimotopes (Clayton, Australia). FM4-64 was purchased from Molecular Probes (Eugene, Oreg.). Tissue culture plastics were from Falcon (Franklin Lakes, N.J.). Penicillin/streptomycin, phosphate buffered salts, foetal calf serum and Minimal Essential Medium (MEM) were obtained from Invitrogen (Carlsbad, Calif.). All other general laboratory chemicals were from Sigma (St. Louis, Mo.) unless otherwise stated.

### 2.1.1 Antibodies and Western Blots

Anti-syndapin I, anti-endophilin I and anti-p85 antibodies were obtained from Santa Cruz Biotechnology (Santa Cruz, Calif.). Anti-Grb2 was purchased from Transduction Laboratory (Lexington, Ky.). Anti-amphiphysin monoclonal antibody was from Pietro De Camilli (Yale, New Haven, Conn.). Anti-dynamin I antibody was as previously described (Tan et al. 2003). Protein samples were separated by SDS-PAGE on 10% or 12% acrylamide gels and transferred to nitrocellulose membrane. Western blots were analysed by the enhanced chemiluminescence method using the SuperSignal West Pico Chemiluminescent Substrate (Pierce, Rockford, Ill.).

### 2.1.2 Immunoprecipitations

Crude (P2) synaptosomes were prepared from rat brain and lysed as described in Example 1.1.3. Synaptosome lysates were incubated with 5 μg of anti-syndapin I or anti-endophilin I antibodies pre-bound to protein G-sepharose (Roche) for 3 h at 4° C. Beads were washed with ice-cold PBS, eluted in SDS-PAGE sample buffer and blotted with anti-syndapin I, anti-endophilin I and anti-dynamin I antibodies.

### 2.1.3 Synaptic Vesicle Recycling Assays

The effect of overexpression of dynamin constructs on synaptic vesicle recycling in CGNs was monitored using the styryl dye FM4-64. Neurons were removed from the culture medium and left to repolarise for 10 min in incubation medium (170 mM NaCl, 3.5 mM KCl, 0.4 mM KH₂PO₄, 20 mM TES (N-tris[hydroxy-methyl]-methyl-2-aminoethane-sulphonic acid), 5 mM NaHCO₃, 5 mM glucose, 1.2 mM Na₂SO₄, 1.2 mM MgCl₂, 1.3 mM CaCl₂, pH 7.4). Neurons were then loaded with FM4-64 by evoking SV recycling using KCl stimulation medium (incubation medium supplemented with 50 mM KCl and 10 μM FM4-64, 50 mM NaCl being removed to maintain osmolarity) for 2 min. Cells were washed with incubation medium and mounted on an epifluorescence microscope (Olympus IX81). After 10 min, FM4-64 was unloaded from the cells by exposure to a 30 s pulse of stimulation medium. Transfected neurons were visualised using a Olympus Plan-Apochromat x40 objective at 480 nm excitation and a band-pass emission filter of 515-555 nm. FM4-64 unloading was monitored at 555 nm excitation and a long pass emission filter of 600 nm. The decrease in fluorescence intensity of FM4-64 loaded nerve terminals on stimulation was visualised using a Hammamatsu Orca-ERG CCD digital camera (Hammamatsu City, Japan) and Metamorph 6.3 imaging software (Molecular Devices, Downingtown, Pa.). The extent of FM4-64 unloading was quantified by normalising the fluorescence value at the start of stimulation to an arbitrary value and monitoring the total decrease in fluorescence on unloading with the KCl stimulus. The kinetics of FM4-64 unloading were estimated by normalising the start and completion of unloading to 1 and 0 respectively. Other methods employed in this study are as described in Example 1.

### 2.2 Results

### 2.2.1 Mapping the Syndapin I Binding Region in Dynamin I

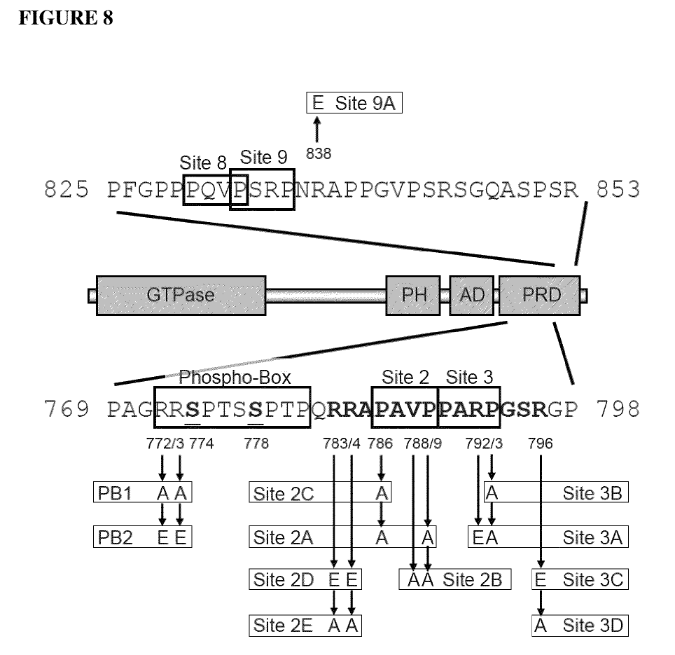

A common characteristic of most binding sites for SH3 domains is the presence of the core amino acid motif PxxP, which is often followed or preceded by a basic Arg residue. There are 13 such motifs in the dynamin I PRD (numbered from site 1 at the N-terminus of the PRD at Pro-753 in dynamin I). Inspection of dynamin I sequence in the PRD reveals two such motifs immediately after the phospho-box, which for convenience are termed site 2 and site 3, (FIG. 8). Since syndapin I binding to dynamin I is regulated by phosphorylation and is known to involve SH3 domain interactions, it was predicted that one of those PxxP motifs forms the core of a binding site for syndapin I. To define the regions by which dynamin I binds syndapin I, a peptide was synthesised corresponding to site 2 and site 3 region (DynI₇₈₃₋₇₉₆-RRAPAVPPARPGSR) (SEQ ID No. 18). Rat brain lysates were incubated with GST-DynI-PRD in the presence of increasing amounts of DynI₇₈₃₋₇₉₆ peptide. Syndapin I binding to DynI-PRD was greatly reduced in a concentration-dependent manner. Syndapin I was identified by mass spectrometry. This was confirmed and expanded by Western blot analysis with anti-syndapin I antibodies. DynI₇₈₃₋₇₉₆ also had a five-fold weaker inhibitory effect on endophilin I binding, but had little effect on binding to p85 (the regulatory subunit of PI-3 kinase), another known SH3-mediated dynamin partner.

Surprisingly, an inhibitory effect of the DynI₇₈₃₋₇₉₆ peptide on amphiphysin I binding was found, although this required 10 times higher peptide concentrations. The binding site of amphiphysin I has previously been mapped to a single site, site 9 (FIG. 8), within the dynamin I-PRD (Grabs et al. 1997; Vallis et al. 1999). Surface plasmon resonance (SPR) studies confirmed a monophasic binding of amphiphysin I-SH3 domain to the isolated C-terminal tail of dynamin I-PRD (Solomaha et al. 2005). However, it is full-length amphiphysin I being detected in the present study. It was thought that the inhibition of amphiphysin I binding could be a non-specific effect of using the arginine- and proline-rich peptide competition approach to study the binding site of a protein. Hence, a site-directed mutagenesis approach was employed to further characterise the binding sites of syndapin I and endophilin I.

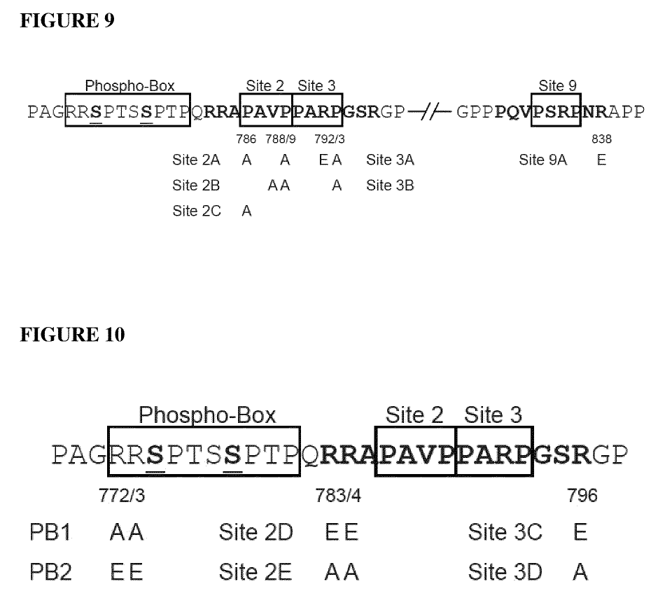

2.2.2 Identification of Core PxxP Syndapin I and Endophilin I Binding Sequences in Dynamin I Since DynI₇₈₃₋₇₉₆ contains two tandem PxxP motifs, ₇₈₆PAVPPARP₇₉₃ (SEQ ID No. 19), specific point mutations in site 2 and site 3 of GST-DynI-PRD were generated to further define the binding sites of both proteins (FIG. 8). Three mutations in the site 2 PAVP (SEQ ID No. 5) sequence were made: a double Pro mutation (termed 2A), a VP mutation (2B) to two alanines, and a single point mutant P786A at the start of site 2 (2C, FIG. 9). All these site 2 mutants greatly reduced binding of both syndapin I and endophilin I. The extent of inhibition of syndapin I binding was the same for all three mutants, but notably endophilin inhibition was less with 2C. Turning to site 3 PARP (SEQ ID No. 20), two mutations were made, an RP mutation to EA (3A) and a point mutation of P793A (3B) (FIG. 9). Both mutations greatly reduced endophilin I binding. The 3B mutant was without effect on syndapin I. When the adjacent basic amino acid was also altered in 3A, syndapin I binding was abolished, suggesting a key role for Arg-792 in syndapin I binding but not Pro-793. It was concluded that syndapin I binds primarily to the core site 2 PAVP (SEQ ID No. 5) motif and that endophilin I binding requires two tandem core motifs encompassing both sites 2 and 3. The results for endophilin I confirm and extend previous observations that its binding may utilise both sites 2 and 3 (Cestra et al. 1999; Ringstad et al. 2001). Sequences outside the PxxP core are explored below.

To determine the specificity of the effect of these PxxP mutants, the binding of amphiphysin I was tested, which has previously been mapped to site 9 by truncation and peptide library approaches (Grabs et al. 1997) and confirmed by a mutagenesis approach (Vallis et al. 1999). As expected, amphiphysin I binding to GST-DynI-9A (R838E) was abolished, but none of the site 2 or site 3 mutants affected its binding. This supports the conclusion that the inhibition of amphiphysin I observed with the DynI₇₈₃₋₇₉₆ peptide was an experimental artefact from the use of arginine- and proline-rich synthetic peptides. Surprisingly, the 9A mutant also showed greatly reduced binding to endophilin I, supporting a previous report of two binding sites for endophilin I in the DynI-PRD by SPR (Solomaha et al. 2005). This suggests the second, low affinity site for endophilin I is site 9. Interestingly, GST-DynI-9A also reduced the binding of syndapin I to a similar extent. The inhibition of syndapin I binding was less than with site 2 mutants, suggestive of a second lower affinity binding site. This data reveals an unexpected secondary binding site for syndapin I at more than 50 amino acids C-terminal to its primary binding site which overlaps with Grb2 and amphiphysin I binding sites (site 8 and 9).

### 2.2.3 Syndapin I and Endophilin I Bind in Either Orientation to Extended Sequences

The extent of the syndapin I and endophilin I binding sites for amino acids beyond the core PxxP was then examined. PxxP motifs bind SH3 domains with a low affinity that is enhanced by other interactions. PxxP motifs form a triangular structure, assuming a left-handed polyproline type II (PPII) helix conformation that fits into the hydrophobic groove of the SH3 domain. Most SH3 domains characterised to date recognise a PxxP core element and a PPII helix. The binding orientation of the SH3 domain is mainly determined by the positively charged amino acids Arg or Lys, positioned N-terminal (class I) or C-terminal (class II) to the core PxxP element (Feng et al. 1994). These basic residues make strong ionic interactions with Asp or Glu, a conserved amino acid found in most SH3 domains. However, some examples of peptide binding in both orientations have been reported, while other peptides have a second PxxP motif in continuation with the core PxxP motif, and these tend to be non-PPII helical (Pisabarro et al. 1998). A PPII helix has a pitch of 3, ie every third amino acid occupies a position on the same side of the helix. Therefore, it is important to examine the role of basic amino acids that may reside in the −3 or +3 position relative to the PxxP core. As is known for amphiphysin I, mutation of such flanking Arg/Lys residues largely abolishes binding. To determine the orientation of syndapin I binding in dynamin I, three such Arg residues were mutated, N-terminal to the site 2 sequence (2D), C-terminal to the core PAVP (3A) (SEQ ID No. 5) and an Arg C-terminal to the site 3 sequence (3C), to negatively charged Glu residues (FIG. 10). As expected, the binding of syndapin I to GST-DynI-2D was abolished. Surprisingly, there was also a strong inhibition of syndapin I binding when two C-terminal basic amino acids, Arg-792, (3A) or Arg-796 (3C) were mutated. To eliminate a potential experimental artefact of introducing negatively charged Glu mutations, the same Arg residues flanking site 2 (2E) and site 3 (3D) were mutated to neutral alanine residues (FIG. 10). As expected, the binding of syndapin I to GST-DynI-2E was also abolished. In addition, the binding of syndapin I to GST-DynI-3D was strongly inhibited. This indicates that syndapin I is able to associate to this region in either orientation, or that its binding extends in both directions over an unusually extended length.

As found with syndapin I, endophilin I binding also appeared to occur in both orientations. Mutation of Arg-783 and Arg-784 (2D and 2E) on the N-terminal side greatly reduced endophilin I binding, while mutation of Arg-796 (3C and 3D) on the C-terminal side of the proline-rich core also significantly reduced binding (FIG. 10).

Surprisingly, mutations of the Arg residues flanking the site 2 (2D) and 3 (3C) into negatively charged Glu residues severely inhibited binding of amphiphysin I (FIG. 10). However, when these Arg residues were substituted with neutral Ala residues, binding of amphiphysin I to either GST-DynI-2E and GST-DynI-3D was normal compared to DynI-WT. Interestingly, introduction of two negative charges in DynI-2D had a slightly stronger but significant inhibitory effect compared to a single negative charge in DynI-3C. This raises the possibility that reversing the charges of Arg residues flanking site 2 and 3 alters the overall folding of the DynI-PRD tertiary structure.

To further define the distinct binding characteristics of syndapin I and endophilin I within the dynamin I PRD, the role of Arg residues in the phospho-box was examined. Mutations of Arg-772 and Arg-773 to Ala (PB1) or Glu residues (PB2) strongly inhibited the binding of syndapin I, but not endophilin I or amphiphysin I. This shows that the two Arg residues in the phospho-box form part of a syndapin I specific binding site. The extension of the syndapin I binding site beyond the canonical site 2 PAVP (SEQ ID No. 5) recognition sequences towards the Arg residues 13 amino acids upstream is an unusual recognition and binding motif for an SH3 domain containing protein.

2.2.4 The Interaction of Dynamin with Syndapin I or Endophilin I is Essential for SVE in Cultured Neurons

Synaptic vesicle turnover in neurons transfected with DynI-WT-GFP was indistinguishable from that in untransfected neurons. However, overexpression of DynI-2B-GFP greatly reduced the amount of FM4-64 dye loaded in nerve terminals compared with untransfected neurons. The extent of synaptic vesicle turnover was quantified by unloading the terminals with a single 50 mM KCl stimulus, which is considered mild. The total synaptic vesicle recycling pool was unaffected in CGNs transfected with DynI-WT-GFP, while after transfection with DynI-2B-GFP it was reduced. The inhibition can be solely accounted for by an effect on SV endocytosis, since there was no effect on the kinetics of dye unloading. Next, the effects of DynI-PB2-GFP and DynI-3B-GFP on SVE were examined. These two mutants were chosen since they specifically fail to bind syndapin I and endophilin I, respectively, providing a clear distinction between them.

The effects of over expressing DynI-9A-GFP, which abolishes amphiphysin I binding, but which also significantly inhibits syndapin I and endophilin I binding was also examined. Quantitative analysis of the stimulated loading and unloading of FM4-64 dye showed that the total synaptic recycling pool was markedly reduced by approximately 30-40% in CGNs transfected with those mutants (59.5±4.3% of control for DynI-PB2-GFP; 56.8±4.4% for DynI-3B-GFP; 58.8±1.7% for DynI-2B-GFP and 71.9±2.7% for DynI-9A-GFP). Again, there was no effect on exocytosis, indicating an inhibition of SVE. Overexpression of DynI-WT-GFP had no significant effect on SVE in CGNs (94.0±5.1% of control.

The results show that the interaction between dynamin I and syndapin I are important for SVE in neurons.

### 2.2.5 Specificity of Inhibition of SVE

Co-pending International Patent Application No. PCT/AU2008/000203 relates to the finding that dynamin II (DynII) is phosphorylated during mitosis and is recruited to the midbody where its dephosphorylation triggers the end of cytokinesis. Inhibiting the dephosphorylation of DynII or blocking its recruitment to the midbody results in multinucleation in mammalian cells. In particular, that application relates to the finding that DynII is phosphorylated at amino acid residue S764 and is dephosphorylated by the Ca²⁺-dependent phosphatase, calcineurin (CaN) and further, that cytokinesis can be inhibited by a phospho-mimetic of this phosphorylated region of DynII. The phospho-mimetic can be peptide mimetic in which at least S764 of DynII or a phosphorylatable amino acid in a homologous position is replaced by a respective negatively charged moiety. The negatively charged moiety can be an amino acid encoded by the genetic code or other amino acid.

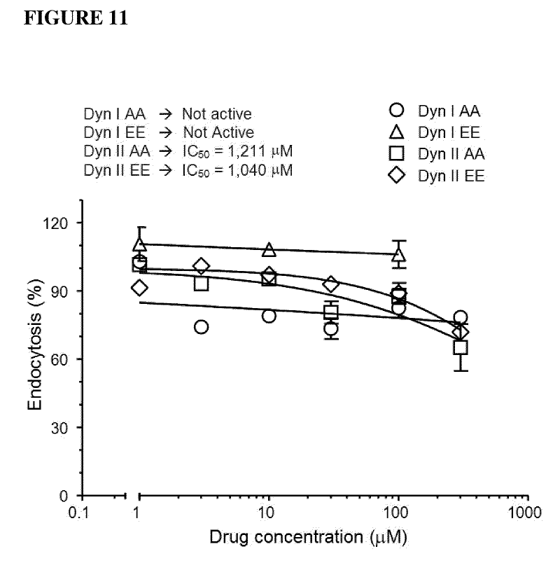

When the DynI₇₆₉₋₇₈₄AA and DynI₇₆₉₋₇₈₄EE peptides were tested for inhibition of RME they were without effect on endocytosis of transferrin (Tfn) in U2OS cells (see FIG. 11). Briefly, non-neuronal U2OS cells were preincubated with vehicle only or each peptide at various concentrations for 15 min. Cells were then incubated with Tfn for 15 min at 37° C., acid washed, fixed and internalized Tfn was detected by fluorescence microscopy. Quantitative analysis of endocytosis in the presence of different concentrations of the peptides is shown as mean fluorescence as a percent of fluorescence in control cells. The results shown in FIG. 11 are representative of 2 independent experiments. Endocytosis in control cells (no peptide) is expressed as 100%.

Previous studies show that homologous phosphobox peptides designed against DynII (DynII1759-775AA or DynII759-775EE) are effective in blocking dynamin II-mediated cytokinesis in HeLa cells, indicating that these penetratin heptamer-linked peptides can work effectively in non-neuronal cell systems (see PCT/AU2008/000203). However, similar to DynI, neither of the two DynII peptide mimetics (AA and EE forms) blocked RME in USOS cells (FIG. 11). Hence, the results reported herein indicate the Dyn I peptide mimetics are targeted specifically to SVE in neurons, suggesting that the DynI phosphobox peptides are relatively neuron-specific in their action.

### 2.3 Discussion

This study defines the syndapin binding sites in the dynamin PRD. By peptide competition in pull-down experiments, syndapin binding was mapped to a region of the PRD encompassed by the Dyn-I peptide ₇₈₃RRAPAVPPARPGSR₇₉₆ (SEQ ID No. 18), which lies immediately after the dynamin phospho-box.

Using a site-directed mutagenesis approach, the syndapin binding site was assigned to the PAVP core motif (SEQ ID No. 5) identified as site 2 within the dynamin PRD. However, the binding involves two features that are unusual for SH3 domain interactions: it extends in both directions from the PxxP core and it involves binding to an unusually extended region of dynamin, completely bridging the phospho-box. Syndapin binding to dynamin exhibited characteristics of both class I and class II binding orientations, both towards and away from the dynamin phospho-box. Mutations of any Arg flanking the site 2 PxxP core motif to a negatively charged residue abolished syndapin binding, including an Arg positioned within the site 3 PxxP core. Mutation of the last proline in site 3 (Pro-793) to alanine did not alter syndapin binding to dynamin, yet double mutation of this Pro and the preceding Arg abolished binding. This suggests that the negative charge in the 3A mutation contributes to the binding inhibition. Further, introduction of negative residues in many locations around this entire region have effects on syndapin binding. For example, mutation of Thr-780 to glutamic acid also blocks syndapin binding (data not shown) as does S774E and S778E (Anggono et al. 2006). However, syndapin does not directly bind to Ser-774, Ser-778 nor Thr-780 as mutations to alanine residues did not alter binding of syndapin (Anggono et al. 2006). Hence, part of DynI of the syndapin SH3 domain bridges or “overhangs” the dynamin phospho-box sequence, without necessarily making direct contact. The two Arg residues at the start of the phospho-box are important for proper recognition and binding of syndapin, despite their location 14 amino acids away from the PxxP core motif. This provides an anchor for the overhang. It is concluded that the unusually extended syndapin binding region stretches from ₇₇₂RRSPTSSPTPQRRAPAVPPARPGSR₇₉₆ (SEQ ID No. 21), with the site 2 PAVP (SEQ ID No. 5) being the core binding site in DynI-PRD (amino acids important for binding are underlined, while amino acids that abolish binding when substituted for Glu but not Ala are in bold). In addition, the syndapin binding site in DynI is sensitive to the introduction of negative charges at almost any point in its length indicating that the syndapin SH3 domain is a highly tuned phospho-sensor.

The endophilin binding region was also defined as tandem site 2 and 3 PAVPPARP (SEQ ID No. 19) core motifs. Like syndapin, endophilin binding had dual characteristics of class I and II orientation. In addition, the PRD sequence ₇₇₈SPTPQ₇₈₂ (SEQ ID No. 22) is an overhang for the endophilin SH3 domain. Mutation of any of these amino acids to alanine does not block its binding to dynamin but binding is inhibited by introduction of a negative charge, S788E in vitro (Anggono et al. 2006). It is concluded that the sequence ₇₇₈SPTPQRRAPAVPPARPGSR₇₉₆ (SEQ ID No. 23) is the endophilin binding region. While endophilin binding does involve an overhang, it does not bridge the whole phospho-box, nor is it N-terminally anchored like syndapin. Hence, the overhang likely makes endophilin a phospho-sensor, but the lack of N-terminal anchor makes it less sensitive than syndapin.

A surprising connection between site 2/3 and 9 was also revealed for amphiphysin binding. Mutations of basic amino acids on either side of the site 2/3 PxxP motifs blocked amphiphysin binding, despite being located more than 50 amino acids away from its binding site. Only mutations to an acidic residue had the effect, suggesting a specific charge interaction is involved. Similarly, a charge alteration at site 9 blocks syndapin and endophilin binding to site 2 and 3. The results indicate long distance interactions between these binding sites are involved in dynamin regulation.

Although the invention has been described with reference to particular embodiments, it will be appreciated by those skilled in the art that numerous variations and/or modifications can be made without departing from the invention as broadly described. The embodiments described are, therefore, to be considered in all respects as illustrative and not restrictive.

### REFERENCES

Aridor, M. & Hannan, L. A. (2000) Traffic jam: a compendium of human diseases that affect intracellular transport processes. Traffic 1, 836-851.
Aridor, M. & Hannan, L. A. (2002) Traffic jams II: an update of diseases of intracellular transport. Traffic 3, 781-790.
Anggono V., Smillie K. J., Graham M. E., Valova V. A., Cousin M. A., and Robinson P. J. (2006) Syndapin I is the phosphorylation-regulated dynamin I partner in synaptic vesicle endocytosis. Nat Neurosci 9, 752-760.
Anggono, V. & Robinson, P. J. (2007) Syndapin I and endophilin I bind to overlapping proline-rich regions of dynamin I: Role in synaptic vesicle endocytosis. J. Neurochem. In Press, DOI 10.1111/j.1471-4159.2007.04574.x.
Cataldo, A. M., Barnett, J. L., Pieroni, C. & Nixon, R. A. (1997) Increased neuronal endocytosis and protease delivery to early endosomes in sporadic Alzheimer's disease: neuropathologic evidence for a mechanism of increased beta-amyloidogenesis., J. Neurosci. 1997, 17:6142.
Cataldo, A., Rebeck, G. W., Ghetri, B., Hulette, C., Lippa, C., Van Broeckhoven, C., van Duijn, C., Cras, P., Bogdanovic, N., Bird, T., Peterhoff, C. & Nixon, R. (2001) Endocytic disturbances distinguish among subtypes of Alzheimer's disease and related disorders. Ann. Neurol. 50, 661-665.
Cao H., Garcia F., and McNiven M. A. (1998) Differential distribution of dynamin isoforms in mammalian cells. Mol Biol Cell 9, 2595-2609.
Cestra G., Castagnoli L., Dente L., Minenkova O., Petrelli A., Migone N., Hoffmuller U., Schneider-Mergener J., and Cesareni G. (1999) The SH3 domains of endophilin and amphiphysin bind to the proline-rich region of synaptojanin 1 at distinct sites that display an unconventional binding specificity. J Biol Chem 274, 32001-32007.
Cloninger, M. J. Biological applications of dendrimers. Curr. Opin. Chem. Biology. 6, 742-748 (2002)
Cousin M. A. and Robinson P. J. (2000a) Ca²⁺ inhibition of dynamin arrests synaptic vesicle recycling at the active zone. J Neurosci 20, 949-957.
Cousin M. A. and Robinson P. J. (2000b) Two mechanisms of synaptic vesicle recycling in rat brain nerve terminals. J Neurochem 75, 1645-1653.
Cousin M. A. and Robinson P. J. (2001) The dephosphins: Dephosphorylation by calcineurin triggers synaptic vesicle endocytosis. Trends Neurosci 24, 659-665.
Di Paolo, G., Sankaranarayanan, S., Wenk, M. R., Daniell, L., Perucco, E., Caldarone, B. J., Flavell, R., Picciotto, M. R., Ryan, T. A., Cremona, O. & De Camilli, P. (2002) Decreased synaptic vesicle recycling efficiency and cognitive deficits in amphiphysin 1 knockout mice. Neuron 33, 789-804.
Dunkley P. R., Jarvie P. E., Heath J. W., Kidd G. J., and Rostas J. A. (1986) A rapid method for isolation of synaptosomes on Percoll gradients. Brain Res 372, 115-129.
Gad, H. et al. Fission and uncoating of synaptic clathrin-coated vesicles are perturbed by disruption of interactions with the SH3 domain of endophilin. Neuron 27, 301-312 (2000).
Feng S., Chen J. K., Yu H., Simon J. A., and Schreiber S. L. (1994) Two binding orientations for peptides to the Src SH3 domain: development of a general model for SH3-ligand interactions. Science 266, 1241-1247.
Floyd S. R., Porro E. B., Slepnev V. I., Ochoa G. C., Tsai L. H., and De Camilli P. (2001) Amphiphysin binds the cdk5 regulatory subunit p35 and is phosphorylated by cdk5 and cdc2. J Biol Chem 276, 8104-8110.
Grabs D., Slepnev V. I., Songyang Z., David C., Lynch M., Cantley L. C., and De Camilli P. (1997) The SH3 domain of amphiphysin binds the proline-rich domain of dynamin at a single site that defines a new SH3 binding consensus sequence. J Biol Chem 272, 13419-13425.
Graham M. E., Anggono V., Bache N., Larsen M. R., Craft G. E., and Robinson P. J. (2007) The in vivo phosphorylation sites of rat brain dynamin I. J Biol Chem. (10.1074/jbc.M609713200).
Humeau, Y., Doussau, F., Grant, N. J. & Poulain, B. (2000) How botulinum and tetanus neurotoxins block neurotransmitter release. Biochimie 82, 427-446.
Itoh T., Erdmann K. S., Roux A., Habermann B., Werner H., and De Camilli P. (2005) Dynamin and the actin cytoskeleton cooperatively regulate plasma membrane invagination by BAR and F-BAR proteins. Dev Cell 9, 791-804.
Lee, C. C., MacKay, J. A., Frechet, J. M. and Szoka, F. C. Designing dendrimers for biological applications. Nature Biotech. 23, 1517-1526 (2005).
Marquez-Sterling, N. R., Lo, A. C., Sisodia, S. S. & Koo, E. H. (1997) Trafficking of cell-surface b-amyloid precursor protein: Evidence that a sorting intermediate participates in synaptic vesicle recycling. J. Neurosci. 17, 140-151.
Metzler, M., Legendre-Guillemin, V., Gan, L., Chopra, V., Kwok, A., McPherson, P. S. & Hayden, M. R. (2001) HIP1 functions in clathrin-mediated endocytosis through binding to clathrin and AP2. J. Biol. Chem. 276, 39271-39276.
Ong, W. Y., Kumar, U., Switzer, R. C., Sidhu, A., Suresh, G., Hu, C. Y. & Patel, S. C. (2001) Neurodegeneration in Niemann-Pick type C disease mice. Exp. Brain Res. 141, 218-231.
Pisabarro M. T., Serrano L., and Wilmanns M. (1998) Crystal structure of the ab1-SH3 domain complexed with a designed high-affinity peptide ligand: implications for SH3-ligand interactions. J Mol Biol 281, 513-521.
Ringstad N., Nemoto Y., and DeCamilli P. (2001) Differential expression of endophilin 1 and 2 dimers at central nervous system synapses. J Biol Chem 276, 40424-40430.
Robinson P. J., Sontag J.-M., Liu J. P., Fykse E. M., Slaughter C., McMahon H. T., and Südhof T. C. (1993) Dynamin GTPase regulated by protein kinase C phosphorylation in nerve terminals. Nature 365, 163-166.
Seto, E. S., Bellen, H. J. & Lloyd, T. E. (2002) When cell biology meets development: endocytic regulation of signaling pathways. Genes Dev. 16, 1314-1336.
Smith, D. S., Niethammer, M., Ayala, R., Zhou, Y., Gambello, M. J., Wynshaw-Boris, A. & Tsai, L. H. (2000) Regulation of cytoplasmic dynein behaviour and microtubule organization by mammalian Lis1. Nat. Cell Biol. 2, 767-775.
Smythies, J. (2000) What is the function of receptor and membrane endocytosis at the postsynaptic neuron? Proc. R. Soc. Lond. Biol. Sci. 267, 1363-1367.
Solomaha E., Szeto F. L., Yousef M. A., and Palfrey H. C. (2005) Kinetics of SH3 domain association with the proline rich domain of dynamins: specificity, occlusion and the effects of phosphorylation. J Biol Chem 280, 23147-23156.
Sung, J. Y., Kim, J., Paik, S. R., Park, J. H., Ahn, Y. S. & Chung, K. C. (2001) Induction of neuronal cell death by Rab5A-dependent endocytosis of alpha-synuclein. J. Biol. Chem. 276, 27441-27448.
Tan T. C., Valova V. A., Malladi C. S., Graham M. E., Berven L. A., Jupp O. J., Hansra G., McClure S. J., Sarcevic B., Boadle R. A., Larsen M. R., Cousin M. A., and Robinson P. J. (2003) Cdk5 is essential for synaptic vesicle endocytosis. Nat Cell Biol 5, 701-710.
Vallis Y., Wigge P., Marks B., Evans P. R., and McMahon H. T. (1999) Importance of the pleckstrin homology domain of dynamin in clathrin-mediated endocytosis. Curr Biol 9, 257-260.
Sadler, K. and Tam, J. P. Peptide dendrimers: applications and synthesis. Rev. Mol. Biotechnology. 90, 195-229, (2002).
Scadding, J. Neuropathic Pain, ACNR•Vol. 3, No. 2 May/June, (2003)
Tomizawa, K. et al. Cophosphorylation of amphiphysin I and dynamin I by Cdk5 regulates clathrin-mediated endocytosis of synaptic vesicles. J. Cell Biol. 163, 813-824 (2003).
Wiffen P, Collins S, McQuay H, et al. Anticonvulsant drugs for acute and chronic pain. Cochrane Database Syst Rev 2000; (3):CD001133 (23 May 2000).

1. An inhibitor of syndapin I binding to dynamin I (Dyn I), the inhibitor being a mimetic of a region of DynI including the serine residues S774 and S778 or phosphorylatable amino acids in homologous positions.

2. A mimetic according to claim 1 being a mimetic of a region of Dyn I comprising amino acid sequence DynI₇₇₂₋₇₈₄ (RRSPTSSPTPQRR) (SEQ ID No. 4) or a homologous sequence thereof.

3. A mimetic according to claim 2 which excludes or does not imitate at least one phosphorylation site provided by S774 or S778 or phosphorylatable amino acids in homologous positions.

4. A mimetic according to claim 3 in which at least one of S774 and S778, or at least one phosphorylatable amino acid in a homologous position to S774 or S778, is substituted for an amino acid other than a negatively charged amino acid.

5. A mimetic according to claim 4 in which both of S774 and S778, or phosphorylatable amino acids in homologous positions, are respectively substituted for an amino acid other than a negatively charged amino acid.

6. A mimetic according to claim 5 which has basic amino acids in positions corresponding to arginine residues R769 and R770, and basic amino acids in positions corresponding to R783 and R784.

7. A mimetic according to claim 6 which retains at least one of R769 and R770 and at least one of R783 and R784, or has arginine amino acid residues in homologous positions to at least one of R769 and R770 and at least one of R783 and R784.

8. A mimetic according to claim 2 having an amino acid sequence identity to DynI₇₇₂₋₇₈₄ (RRSPTSSPTPQRR) (SEQ ID No. 4) of 60% or greater.

9. A mimetic according to claim 8 being a mimetic of the amino acid sequence consisting of DynI₇₇₂₋₇₈₄ (RRSPTSSPTPQRR) (SEQ ID No. 4).

10. A mimetic according to claim 1 being a peptide mimetic up to 40 amino acids in length.

11. A mimetic according to claim 1 adapted to pass across outer cell membrane into neurons or being coupled to a facilitator moiety for facilitating passage or translocation of the mimetic into neurons.

12. A mimetic according to claim 11 coupled to a penetratin amino acid sequence for facilitating passage of the mimetic into neurons.

13. A pharmaceutical composition comprising at least one inhibitor of syndapin I binding to dynamin I (Dyn I) together with a pharmaceutically acceptable carrier, the inhibitor being a mimetic of a region of DynI including the serine residues S774 and S778 or phosphorylatable amino acids in homologous positions.

14. A method for prophylaxis or treatment of a neurological disease or condition in an individual, comprising administering to the individual an effective amount of an inhibitor of syndapin I binding to dynamin I (Dyn I), the inhibitor being a fragment of a region of DynI including the serine residues S774 and S778 or phosphorylatable amino acids in homologous positions, or a mimetic of the region of Dyn I.

15. A method according to claim 14, the inhibitor being a mimetic of a region of Dyn I comprising amino acid sequence DynI₇₇₂₋₇₈₄ (RRSPTSSPTPQRR) (SEQ ID No. 4) or a homologous sequence thereof.

16. A method according to claim 15 wherein the mimetic excludes or does not imitate at least one phosphorylation site provided by S774 or S778 or phosphorylatable amino acids in homologous positions.

17. A mimetic according to claim 16 in which at least one of S774 and S778, or at least one phosphorylatable amino acid in a homologous position to S774 or S778, is substituted for an amino acid other than a negatively charged amino acid.

18. A mimetic according to claim 17 in which both of S774 and S778 or phosphorylatable amino acids in homologous positions are respectively substituted for an amino acid other than a negatively charged amino acid.

19. A mimetic according to claim 16 which has basic amino acids in positions corresponding to arginine residues R769 and R770, and basic amino acids in positions corresponding to R783 and R784.

20. A mimetic according to claim 16 which retains at least one of R769 and R770 and at least one of R783 and R784, or has arginine amino acid residues in homologous positions to at least one of R769 and R770 and at least one of R783 and R784.

21. A mimetic according to claim 15 having an amino acid sequence identity to DynI₇₇₂₋₇₈₄ (RRSPTSSPTPQRR) (SEQ ID No. 4) of 60% or greater.

22. A mimetic according to claim 21 being a mimetic of the amino acid sequence consisting of DynI₇₇₂₋₇₈₄ (RRSPTSSPTPQRR) (SEQ ID No. 4).

23. A method according to claim 14 wherein the mimetic is a peptide mimetic up to 40 amino acids in length.

24. A method according to claim 14 wherein the mimetic is adapted to pass across outer cell membrane into neurons or is coupled to a facilitator moiety which facilitates passage or translocation of the mimetic into neurons.

25. A method according to claim 14 wherein the neurological disease or condition is selected from the group consisting of diseases and conditions associated with cell vesicle trafficking, diseases and conditions associated with synaptic signal transmission, psychotic and psychiatric conditions, neurodegenerative diseases, epilepsy, neuropathic pain, β-amyloid associated diseases, Alzheimer's disease, Parkinson's disease, Huntington's disease, Lewis body dementias and neuroparalytic diseases.

26. A method according to claim 25 wherein the neurological disease or condition is epilepsy.

27. A method for prophylaxis or treatment of a neurological disease or condition in a mammal, comprising administering to the mammal an effective amount of an inhibitor of syndapin I binding to dynamin I.

28. A method for prophylaxis or treatment of epilepsy in a mammal, comprising administering to the mammal an effective amount of an inhibitor of syndapin I binding to dynamin I.

29. A method for inhibiting synaptic signal transmission, comprising treating a neuron with an effective amount of an inhibitor of syndapin I binding to dynamin I.

30. A method for inhibiting synaptic vesicle endocytosis, comprising treating a neuron with an effective amount of an inhibitor of syndapin I binding to dynamin I.

31. A method for screening a test compound for capacity to inhibit interaction of dynamin I (DynI) with syndapin I, comprising:
providing the test compound to be screened;
incubating the test compound with DynI or a molecule substituting for DynI in the presence of a syndapin I or a molecule substituting for syndapin I; and
determining whether the compound inhibits the interaction of DynI with syndapin I.

32. A method according to claim 31 wherein the determination of whether the test compound inhibits the interaction of Dyn I with syndapin I involves determining whether or not synaptic vesicle endocytosis is inhibited by the compound.
